SEQUENCE LISTING (56 sequences)

SEQ ID NO: 1 - protein, 178 aa, human
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Artificial Sequence
  lipocalin mutein
QDSTSDLIPAPPLSKVPLQQNFQDNQFHGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - protein, 272 aa, human
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 15 aa, Artificial Sequence
  linker
GGGGSGGGGSGGGGS

SEQ ID NO: 5 - protein, 18 aa, Artificial Sequence
  linker
PSTPPTNSSSTPPTPSPS

SEQ ID NO: 6 - protein, 14 aa, Artificial Sequence
  linker
GGSGNSSGSGGSPV

SEQ ID NO: 7 - protein, 20 aa, Artificial Sequence
  linker
ASPAAPAPASPAAPAPSAPA

SEQ ID NO: 8 - protein, 66 aa, Artificial Sequence
  linker
AGSGGSGGSGGSPVPSTPPTPSPSTPPTPSPSGGSGNSSGSGGSPVPSTPPTPSPSTPPTPSPSAS

SEQ ID NO: 9 - protein, 32 aa, Artificial Sequence
  linker
PSTPPTPSPSTPPTPSPSGGSGNSSGSGGSPV

SEQ ID NO: 10 - protein, 74 aa, Artificial Sequence
  linker
AGSGGSGGSGGSPVPSTPPTNSSSTPPTPSPSPVPSTPPTNSSSTPPTPSPSPVPSTPPTNSSSTPPTPSPSAS

SEQ ID NO: 11 - protein, 40 aa, Artificial Sequence
  linker
ASPAAPAPASPAAPAPSAPAASPAAPAPASPAAPAPSAPA

SEQ ID NO: 12 - protein, 10 aa, Artificial Sequence
  linker
VDDIEGRMDE

SEQ ID NO: 13 - protein, 11 aa, Artificial Sequence
  linker
ENLYFQGRMDE

SEQ ID NO: 14 - protein, 10 aa, Artificial Sequence
  linker
GGGGSGGGGS

SEQ ID NO: 15 - protein, 327 aa, Artificial Sequence
  antibody heavy chain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 214 aa, Artificial Sequence
  antibody light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 228 aa, Artificial Sequence
  antibody Fc region
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 228 aa, Artificial Sequence
  antibody Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 228 aa, Artificial Sequence
  antibody Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 20 - protein, 228 aa, Artificial Sequence
  antibody Fc region
ESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYNVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 22 - protein, 178 aa, Artificial Sequence
  lipocalin mutein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 23 - protein, 178 aa, Artificial Sequence
  lipocalin mutein
QDSTSDLIPAPPLSKVPLQQNFQDNQFHGKWYVVGQAGNIRLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 24 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 25 - protein, 175 aa, Artificial Sequence
  lipocalin mutein
QDSTSDLIPAPPLSKVPLQQNFQDNQFHGKWYVVGQAGNIKLREDSKMMATIYELKEDKSYDVTGVSFDDKKCTYAIMTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 26 - protein, 178 aa, Artificial Sequence
  lipocalin mutein
QDSTSDLIPAPPLSKVPLQQNFQDNQFHGKWYVVGQAGNIKLREDKDPVKMMATIYELKEDKSYDVTGVTFDDKKCRYDISTFVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 27 - protein, 178 aa, Artificial Sequence
  lipocalin mutein
QDSTSDLIPAPPLSKVPLQQNFQDNQFHGKWYVVGQAGNIRLREDKDPHKMMATIYELKEDKSYDVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 28 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYDVTGVTFDDKKCTYAISTLVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 29 - protein, 178 aa, Artificial Sequence
  lipocalin mutein
QDSTSDLIPAPPLSKVPLQQNFQDNQFHGKWYVVGQAGNIRLREDKDPSKMMATIYELKEDKSYDVTAVTFDDKKCNYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 30 - protein, 178 aa, Artificial Sequence
  lipocalin mutein
QDSTSDLIPAPPLSKVPLQQNFQDNQFHGKWYVVGMAGNNLLREDKDPHKMSATIYELKEDKSYNVTDVMFLDKKCQYIIWTFVPGSQPGEFTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 31 - protein, 178 aa, Artificial Sequence
  lipocalin mutein
QDSTSDLIPAPPLSKVPLQQNFQDNQFQGKWYVVGMAGNNLLREDKDPHKMSATIYELKEDKSYNVTDVMFLDKKCQYIIWTFVPGSQPGELTLGFIRSDLGHTSYLVRVVSTNYNQHAMVFFKSVIQNREWFGITLYGRTKELTSELKENFIRFSKSLGLPENHIVFPVPIDQCIDG

SEQ ID NO: 32 - protein, 178 aa, Artificial Sequence
  lipocalin mutein
QDSTSDLIPAPPLSKVPLQQNFQDYQFQGKWYVVGMAGNNLLREDKDPHKMGATIYELKEDKSYNVTDVMLLDKKCQYIIQTFVPGSQPGESTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 33 - protein, 178 aa, Artificial Sequence
  lipocalin mutein
QDSTSDLIPAPPLSKVPLQQNFQDNQFQGKWYVVGMAGNNLLREDKDPHKMGATIYELKEDKSYNVTDVMFLDKKCQHIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 34 - protein, 178 aa, Artificial Sequence
  lipocalin mutein
QDSTSDLIPAPPLSKVPLQQNFQDDQFQGKWYVVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 35 - protein, 178 aa, Artificial Sequence
  lipocalin mutein
QDSTSDLIPAPPLSKVPLQQNFQDNQFQGKWYIVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 36 - protein, 178 aa, Artificial Sequence
  lipocalin mutein
QDSTSDLIPAPPLSKVPLQRNFQDNQFQGKWYVVGMAGNNLLRVDKDPHKMGATIYELKEDKSYNVTDVMFLDKKCQYIIWTFVPGSQPGELTLGFIKSDPGHTSYLVRVVSTNYNQHAMVYFKSVIQNREWFGITLYGRTKELTSELKENFIRFSKSLGLPENHIVFPVPIDQCIDG

SEQ ID NO: 37 - protein, 178 aa, Artificial Sequence
  lipocalin mutein
QDSTSDLIPAPPLSKVPLQQNFQDNQFQGKWYVVGMAGNNLLREDKDPHKMSATIYELKEDKSYNVTDVMFLDKKCQYINWPFVPGSQPGEFTLGFIKSDLGPTSYLVRVVSTNYNQHAMVFFKSVIQNREWFGITLYGRTKELTSELKENFIRFSKSLGLPENHIVFPVPIDQCIDG

SEQ ID NO: 38 - protein, 178 aa, Artificial Sequence
  lipocalin mutein
QDSTSDLIPAPPLSKVPLQQNFQDNQFQGKWYVVGMAGNNLLREDKDPHKMGATIYELNEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 39 - protein, 178 aa, Artificial Sequence
  lipocalin mutein
QDSTSDLIPAPPLSKVPLQQNFQDNQFQGKWYVVGMAGNNLLRDDKDPHKMSATIYELKEDKSYNVTDVMLLDKKCHYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 40 - protein, 10 aa, Artificial Sequence
  antibody CDR
GFNIKDTYIH

SEQ ID NO: 41 - protein, 18 aa, Artificial Sequence
  antibody CDR
RIYPTNGYTRYADSVKGR

SEQ ID NO: 42 - protein, 11 aa, Artificial Sequence
  antibody CDR
WGGDGFYAMDY

SEQ ID NO: 43 - protein, 12 aa, Artificial Sequence
  antibody CDR
RASQDVNTAVAW

SEQ ID NO: 44 - protein, 7 aa, Artificial Sequence
  antibody CDR
SASFLYS

SEQ ID NO: 45 - protein, 9 aa, Artificial Sequence
  antibody CDR
QQHYTTPPT

SEQ ID NO: 46 - protein, 120 aa, Artificial Sequence [sequence omitted]

SEQ ID NO: 47 - protein, 107 aa, Artificial Sequence [sequence omitted]

SEQ ID NO: 48 - protein, 450 aa, Artificial Sequence
  antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 447 aa, Artificial Sequence
  antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 640 aa, Artificial Sequence
  fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 52 - protein, 407 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 53 - protein, 640 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 407 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 449 aa, Artificial Sequence
  antibody heavy chain
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 219 aa, Artificial Sequence
  antibody light chain
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0366884 A1
(43) Pub. Date: Nov. 16, 2023

(54) BIOMARKER METHODS AND USES

(71) Applicant: Pieris Pharmaceuticals GmbH, Hallbergmoos (DE)

(72) Inventors: Markus ZETTL, Hallbergmoos (DE); Aizea Morales KASTRESANA, Hallbergmoos (DE); Cornelia WURZENBERGER, Hallbergmoos (DE); Janet PEPER-GABRIEL, Hallbergmoos (DE); Marleen RICHTER, Hallbergmoos (DE); Nicole ANDERSEN, Hallbergmoos (DE); Rachida BEL AIBA, Hallbergmoos (DE); Andrea ALLERSDORFER, Hallbergmoos (DE); Verena NODERER, Hallbergmoos (DE)

(21) Appl. No.: 18/044,908

(22) PCT Filed: Sep. 17, 2021
(86) PCT No.: PCT/EP2021/075629

§ 371 Date: Mar. 10, 2023

Related U.S. Application Data
(60) Provisional application No. 63/164,808, filed on Mar. 23, 2021.
(60) Provisional application No. 63/080,361, filed on Sep. 18, 2020.

(51) Int. Cl. G01N 33/574; C07K 16/32; A61P 35/00

(52) U.S. Cl. CPC G01N 33/574; C07K 16/32; A61P 35/00; G01N 2800/52; G01N 2333/70578; C07K 2319/00

(57) ABSTRACT

The disclosure provides methods for predicting a positive clinical outcome for a cancer patient upon treatment with a 4-1BB agonistic agent, for assessing activity of such 4-1BB agonistic agent in a subject, preferably a cancer patient, and for selecting a dose of a 4-1BB agonistic agent for treating a disease, such as cancer. The disclosure also provides methods of treating cancer as well as uses of biomarkers and uses of kits for their detection.

### 1. BACKGROUND

4-1BB is a co-stimulatory immune checkpoint and member of the tumor necrosis factor receptor (TNFR) family. It is primarily expressed on activated CD4+ and CD8+ T cells, activated B cells, and natural killer (NK) cells, and plays an important role in the regulation of the immune response. The clustering of 4-1BB leads to activation of the receptor and downstream signaling (Yao et al., 2013, Snell et al., 2011). In a T cell pre-stimulated by the T cell receptor (TCR) binding to a cognate major histocompatibility complex (MHC) target, co-stimulation via 4-1BB leads to enhanced activation, survival, and proliferation, as well as to the production of pro-inflammatory cytokines and an improved capacity to kill (Dawicki and Watts, 2004, Lee et al., 2002).

4-1BB agonistic agents are currently being tested in the clinic, and there is the need to identify suitable biomarkers, e.g., biomarkers associated with beneficial clinical outcomes.

### II. SUMMARY

The present disclosure provides, among other things, methods for predicting a positive clinical outcome for a cancer patient upon treatment with a 4-1BB agonistic agent and for assessing activity of such 4-1BB agonistic agent in a subject, preferably a cancer patient. The disclosure also provides methods of treating cancer as well as uses of biomarkers and of kits for their detection.

More particularly, the present disclosure relates to methods and uses involving soluble 4-1BB (s4-1BB).

### III. DEFINITIONS

The following list defines terms, phrases, and abbreviations used throughout the instant specification. All terms listed and defined herein are intended to encompass all grammatical forms.

As used herein, unless otherwise specified, “4-1BB” means human 4-1BB (hu4-1BB). Human 4-1BB means a full-length protein defined by UniProt Q07011, a fragment thereof, or a variant thereof. 4-1BB is also known as CD137, tumor necrosis factor receptor superfamily member 9 (TNFRSF9) and induced by lymphocyte activation (ILA). In some particular embodiments, 4-1BB of non-human species, e.g., cynomolgus 4-1BB and mouse 4-1BB, is used.

The term “soluble 4-1BB (s4-1BB)” refers to a soluble (i.e., non-cell membrane bound) form of 4-1BB, which may be released from activated lymphocytes. The term “circulating s4-1BB”, as used herein, refers to s4-1BB circulating in the blood stream. Methods to measure s4-1BB in a biological sample (such as blood serum or blood plasma), e.g., by means of an enzyme-linked immunosorbent assay (ELISA), are known to a person skilled in the art, and are described, for example, in Segal et al., 2018. Corresponding kits are also commercially available, e.g., from Invitrogen/Thermo Fisher (“CD137 (4-1BB) (Soluble) Human ELISA Kit”) or BioLegend (“LEGEND MAX™ Human Soluble CD137/4-1BB ELISA Kit”).

As used herein, unless otherwise specified, the term “s4-1BB level” refers to the absolute level (e.g., given in weight per volume unit of the (fluid) biological sample, e.g., in pg/ml), area under the curve (AUC) over a specified period of time or relative level (e.g., relative to a baseline, such as the level of s4-1BB in the biological sample prior to treatment with the 4-1BB agonistic agent) of s4-1BB in a biological sample, e.g., blood serum or blood plasma.

The term “4-1BB agonistic agent”, as used herein, refers to an agent being able to activate 4-1BB and the 4-1BB signaling pathway. A 4-1BB agonistic agent may specifically bind to 4-1BB.

As used herein, unless otherwise specified, “HER2” means human HER2 (huHER2). Human HER2 means a full-length protein defined by UniProt P04626, a fragment thereof, or a variant thereof. HER2 is also known as human epidermal growth factor receptor 2, HER2/neu, receptor tyrosine-protein kinase erbB-2, cluster of differentiation 340 (CD340), proto-oncogene Neu, ERBB2 (human), Erbb2 (rodent), c-neu, or p185. Human HER2 is encoded by the ERBB2 gene. In some particular embodiments, HER2 of non-human species, e.g., cynomolgus HER2 and mouse HER2, is used.

The term “anti-” or “targeting”, when used to describe a molecule in association with a protein target of interest (e.g., 4-1BB or HER2), means the molecule is capable of binding the protein target and/or modulating one or more biological functions of the protein target. For example, an “anti-4-1BB” molecule as described herein, is capable of binding 4-1BB and/or modulating one or more biological functions of 4-1BB. “Biological function” of a protein target refers to the ability of the protein target to carry out its biological mission(s), e.g., binding to its binding partner(s) and mediating signaling pathway(s).

As used herein, “T cell activation” refers to a process leading to proliferation and/or differentiation of T cells. The activation of T cells may lead to the initiation and/or perpetuation of immune responses. As used herein, T cell activation may be used to assess the health of subjects with disease or disorders associated with dysregulated immune responses, such as cancer, autoimmune disease, and inflammatory disease. T cell proliferation refers to the expansion of a T cell population. “T cell proliferation” and “T cell expansion” are used interchangeably herein.

The terms “enhance T cell activity”, “activate T cells”, and “stimulate T cell response”, are used interchangeably herein and refer to inducing, causing, or stimulating T cells to have sustained or amplified biological functions, or renew or reactivate exhausted or inactive T cells. Exemplary signs of enhanced T cell activity include, but are not limited to: increased secretion of interleukin-2 (IL-2) from T cells, increased secretion of Interferon-gamma (IFN-γ) from T cells, increased T cell proliferation, and/or increased antigen responsiveness (e.g., viral, pathogen, and tumor clearance). Methods of measuring such enhancement are known to the skilled in the art.

“Cancer” and “cancerous” refers to the physiological condition in mammals that is typically characterized by unregulated cell growth. A “tumor” may comprise one or more cancerous cells. A “lesion” is a localized change in a tissue or an organ. Tumors are types of lesions. “Target lesions” are lesions that have been specifically measured. “Non-target lesions” are lesions whose presences have been noted, but whose measurements have not been taken. The terms “cancer”, “tumor”, and “lesion” are used interchangeably herein. The cancer may be selected from the group consisting of gastric cancer, gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer, colon cancer, pancreatic cancer, biliary tract cancer, salivary duct cancer, bladder cancer, and cancer of unknown primary. The cancer may be a HER2-expressing tumor/cancer.

As used herein, the term “HER2-expressing tumor” is meant to refer to a tumor with detectable expression of HER2, e.g., detectable by a quantitative assay, such as an mRNA-based qRT-PCR assay. In some embodiments, the term “HER2-expressing tumor” refers to a HER2-positive (HER2+) tumor or to a tumor characterized by a low expression of HER2.

As used herein, the term “HER2-positive (HER2+) tumor” is meant to refer to a tumor which is classified as a HER2+ tumor by immunohistochemistry (IHC) and/or (fluorescent) in situ hybridization ((F)ISH) analysis, e.g., according to the 2018 ASCO/CAP guidelines for HER2 testing in breast cancer (Wolff et al., 2018) or the 2016 CAP/ASCP/ASCO guidelines for HER2 testing in gastric or gastroesophageal adenocarcinoma (Bartley et al., 2016). In some particular embodiments, a HER2+ tumor is characterized by a HER2 status of IHC3+, IHC2+/(F)ISH+ or (F)ISH+, preferably IHC3+ or IHC2+/(F)ISH+. In some embodiments, a HER2+ tumor is characterized by HER2 gene amplification, e.g., as determined by (F)ISH or next generation sequencing (NGS) analysis.

A “tumor characterized by a low expression of HER2” (also referred to herein as “HER2 low tumor”) refers to a tumor which exhibits expression of HER2, albeit at a level which does not warrant its classification as a HER2+ tumor by IHC and (F)ISH. In some embodiments, a HER2 low tumor is a tumor which exhibits expression of HER2 at a level which is detectable by a quantitative assay, such as an mRNA-based qRT-PCR assay, but which is not classified as a HER2+ tumor by IHC and/or (F)ISH, e.g., according to the 2018 ASCO/CAP guidelines for HER2 testing in breast cancer (Wolff et al., 2018) or the 2016 CAP/ASCP/ASCO guidelines for HER2 testing in gastric or gastroesophageal adenocarcinoma (Bartley et al., 2016). In some particular embodiments, a HER2 low tumor is characterized by a HER2 status of IHC1+ or IHC2+/(F)ISH−. However, for the avoidance of doubt, HER2 low tumors may also include tumors that are, for example, characterized by a HER2 status of IHC0 (and (F)ISH−), but that still exhibit expression of HER2, e.g., as determined in a quantitative assay, such as an mRNA-based qRT-PCR assay. In some embodiments, a HER2 low tumor does not exhibit HER2 gene amplification, e.g., as determined by (F)ISH or next generation sequencing (NGS) analysis.

The term “metastatic” refers to a state of cancer where the cancer cells break away from where they first formed and form new tumors (metastatic tumors) in other parts of the body. An “advanced” cancer may be locally advanced or metastatic. Locally advanced cancer refers to cancer that has grown outside the site or organ of origin but has not yet spread to distant parts of the body.

“Tumor microenvironment (TME)” refers to the environment around a tumor, composed of non-cancer cells and their stroma. The tumor stroma comprises a compilation of cells, including fibroblasts/myofibroblasts, glial, epithelial, fat, immune, vascular, smooth muscle, and immune cells, blood vessels, signaling molecules, and the extracellular matrix (ECM), and serves a structural or connective role. In this context, “full tumor tissue” consists of tumor cells and tumor stroma.

As used herein, an “anti-tumor agent” or “anti-tumor drug” may act on a tumor, particularly a malignant tumor, and preferably has an anti-tumor effect or anti-tumor activity. The “anti-tumor effect” or “anti-tumor activity” refers to actions of an anti-tumor agent on a tumor, particularly a malignant tumor, including stimulation of tumor-specific immune responses, reduction in target lesion, reduction in tumor size, suppression of the growth of tumor cells, suppression of metastasis, complete remission, partial remission, stabilization of disease, extension of the term before recurrence, extension of survival time of patients, or improvement of quality of life of patients.

As used herein, “treat” or “treatment” refers to clinical intervention designed to alter the natural course of the subject being treated during the course of a physiological condition or disorder or clinical pathology. A treatment may be a therapeutic treatment and/or a prophylactic or preventative measure, wherein the object is to prevent or slow down (lessen) an undesired physiological change or disorder, such as the growth, development or spread of a hyperproliferative condition, such as cancer. Desired effects of treatment include, but are not limited to, decreasing the rate of disease progression, ameliorating or palliating the disease state, alleviating symptoms, stabilizing or not worsening the disease state, and remission of improved prognosis, whether detectable or undetectable. Desired effects of treatment also include prolonging survival as compared to expected survival if not receiving treatment. A subject in need of treatment includes a subject already with the condition or disorder or prone to have the condition or disorder or a subject in which the condition or disorder is to be prevented.

A treatment given to a subject with tumor may lead to tumor response as described in Response Evaluation Criteria in Solid Tumors (RECIST) guideline (version 1.1) (Eisenhauer et al., 2009). For example, a treatment given to a subject with tumor may lead to complete response, partial response, stable disease, or progressive disease. “Complete response (CR)” refers to the disappearance of all target lesions. “Partial response (PR)” refers to at least a 30% decrease in the sum of diameters of target lesions, taking as reference the baseline sum diameters. “Progressive disease (PD)” refers to At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. “Stable disease (SD)” refers to neither sufficient shrinkage to qualify for PR nor sufficient increase to qualify for PD, taking as reference the smallest sum diameters while on study. “Duration of response (DoR)” may be calculated as the time from the date of first documented response (CR or PR) to the date of documented progression or death after achieving response.

An “effective amount” of a drug or therapeutic agent is an amount sufficient to effect beneficial or desired effects of a treatment. For example, an effective amount an anti-tumor agent may be one that is sufficient to enhance T cell activation to a desired level. In some embodiments, the effectiveness of a drug or therapeutic agent can be determined by suitable methods known in the art. For example, the effectiveness of an anti-tumor agent may be determined by Response Evaluation Criteria in Solid Tumors (RECIST). An effective amount can be administered in one or more individual administrations or doses. An effective amount can be administered alone with one agent or in combination with one or more additional agents.

As used herein, “antibody” includes whole antibodies or any antigen binding fragment (i.e., “antigen-binding domain”) or single chain thereof. A whole antibody refers to a glycoprotein comprising at least two heavy chains (HCs) and two light chains (LCs) inter-connected by disulfide bonds. Each heavy chain is comprised of a heavy chain variable domain (V_{H} or HCVR) and a heavy chain constant region (C_{H}). The heavy chain constant region is comprised of three domains, C_{H}1, C_{H2} and C_{H}3. Each light chain is comprised of a light chain variable domain (V_{L} or LCVR) and a light chain constant region (C_{L}). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen (for example, PD-L1). The constant regions of the antibodies may optionally mediate the binding of the immunoglobulin to host tissues or factors, including various cells of the immune system (e.g., effector cells) and the first component (C1q) of the classical complement system.

As used herein, “antigen-binding domain” or “antigen-binding fragment” of an antibody refers to one or more fragments of an antibody that retain the ability to specifically bind to an antigen (e.g., HER2). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H}1 domains; (ii) a F(ab′)₂ fragment comprising two Fab fragments linked by a disulfide bridge at the hinge region; (iii) a Fab′ fragment consisting of the V_{H}, V_{L}, C_{L} and C_{H}1 domains and the region between C_{H}1 and C_{H2} domains; (iv) an Fd fragment consisting of the V_{H} and C_{H}1 domains; (v) a single-chain Fv fragment consisting of the V_{H} and V_{L} domains of a single arm of an antibody, (vi) a dAb fragment (Ward et al., 1989) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., 1993); (ix) a “domain antibody fragment” containing only the V_{H} or V_{L}, where in some instances two or more V_{H} regions are covalently joined.

Antibodies may be polyclonal or monoclonal; xenogeneic, allogeneic, or syngeneic; or modified forms thereof (e.g., humanized, chimeric, or multispecific). Antibodies may also be fully human.

The term “effector functions” as used herein with respect to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the term “lipocalin” refers to a monomeric protein of approximately 18-20 kDa in weight, having a cylindrical p-pleated sheet supersecondary structural region comprising a plurality of p-strands (preferably eight p-strands designated A to H) connected pair-wise by a plurality of (preferably four) loops at one end to thereby comprise a ligand-binding pocket and define the entrance to the ligand-binding pocket. Preferably, the loops comprising the ligand-binding pocket used in the present invention are loops connecting the open ends of p-strands A and B, C and D, E and F, and G and H, and are designated loops AB, CD, EF, and GH. It is well-established that the diversity of the said loops in the otherwise rigid lipocalin scaffold gives rise to a variety of different binding modes among the lipocalin family members, each capable of accommodating targets of different sizes, shape, and chemical character (reviewed, e.g. in Skerra, 2000, Flower et al., 2000, Flower, 1996). It is understood that the lipocalin family of proteins has naturally evolved to bind a wide spectrum of ligands, sharing unusually low levels of overall sequence conservation (often with sequence identities of less than 20%) yet retaining a highly conserved overall folding pattern. The correspondence between positions in various lipocalins is also well-known to one of skill in the art (see, e.g., U.S. Pat. No. 7,250,297). Proteins falling in the definition of “lipocalin” as used herein include, but are not limited to, human lipocalins including tear lipocalin (Tlc, Lcn1), Lipocalin-2 (Lcn2) or neutrophil gelatinase-associated lipocalin (NGAL), apolipoprotein D (ApoD), apolipoprotein M, α₁-acid glycoprotein 1, α₁-acid glycoprotein 2, α₁-microglobulin, complement component 8γ, retinol-binding protein (RBP), the epididymal retinoic acid-binding protein, glycodelin, odorant-binding protein IIa, odorant-binding protein IIb, lipocalin-15 (Lcn15), and prostaglandin D synthase.

As used herein, “Lipocalin-2” or “neutrophil gelatinase-associated lipocalin” refers to human Lipocalin-2 (hLcn2) or human neutrophil gelatinase-associated lipocalin (hNGAL) and further refers to the mature human Lipocalin-2 or mature human neutrophil gelatinase-associated lipocalin. The term “mature” when used to characterize a protein means a protein essentially free from the signal peptide. A “mature hNGAL” of the instant disclosure refers to the mature form of human neutrophil gelatinase-associated lipocalin, which is free from the signal peptide. Mature hNGAL is described by residues 21-198 of the sequence deposited with the SWISS-PROT Data Bank under Accession Number P80188, the amino acid sequence of which is indicated in SEQ ID NO: 1.

As used herein, a “native sequence” refers to a protein or a polypeptide having a sequence that occurs in nature or having a wild-type sequence, regardless of its mode of preparation. Such native sequence protein or polypeptide can be isolated from nature or can be produced by other means, such as by recombinant or synthetic methods.

The “native sequence lipocalin” refers to a lipocalin having the same amino acid sequence as the corresponding polypeptide derived from nature. Thus, a native sequence lipocalin can have the amino acid sequence of the respective naturally-occurring (wild-type) lipocalin from any organism, in particular, a mammal. The term “native sequence”, when used in the context of a lipocalin specifically encompasses naturally-occurring truncated or secreted forms of the lipocalin, naturally-occurring variant forms such as alternatively spliced forms and naturally-occurring allelic variants of the lipocalin. The terms “native sequence lipocalin” and “wild-type lipocalin” are used interchangeably herein.

As used herein, a “mutein,” a “mutated” entity (whether protein or nucleic acid), or “mutant” refers to the exchange, deletion, or insertion of one or more amino acids or nucleotides, compared to the naturally-occurring (wild-type) protein or nucleic acid. Said term also includes fragments of a mutein as described herein. The present disclosure explicitly encompasses lipocalin muteins, as described herein, having a cylindrical p-pleated sheet supersecondary structural region comprising eight p-strands connected pair-wise by four loops at one end to thereby comprise a ligand-binding pocket and define the entrance of the ligand-binding pocket, wherein at least one amino acid of each of at least three of said four loops has been mutated as compared to the native sequence lipocalin. Lipocalin muteins of the present disclosure preferably have the function of binding 4-1BB as described herein.

As used herein, the term “fragment,” in connection with the lipocalin muteins of the disclosure, refers to proteins or polypeptides derived from full-length mature hNGAL or lipocalin muteins that are N-terminally and/or C-terminally truncated, i.e., lacking at least one of the N-terminal and/or C-terminal amino acids. Such fragments may include at least 10 or more, such as 20 or 30 or more, consecutive amino acids of the primary sequence of mature hNGAL or the lipocalin mutein it is derived from and are usually detectable in an immunoassay of mature hNGAL. Such a fragment may lack up to 2, up to 3, up to 4, up to 5, up to 10, up to 15, up to 20, up to 25, or up to 30 (including all numbers in between) of the N-terminal and/or C-terminal amino acids. It is understood that the fragment is preferably a functional fragment of mature hNGAL or the lipocalin mutein from which it is derived, which means that it preferably retains the binding specificity, preferably to 4-1BB, of mature hNGAL or the lipocalin mutein it is derived from. As an illustrative example, such a functional fragment may comprise at least amino acids at positions 13-157, 15-150, 18-141, 20-134, 25-134, or 28-134 corresponding to the linear polypeptide sequence of mature hNGAL.

A “fragment” with respect to 4-1BB or HER2 refers to N-terminally and/or C-terminally truncated 4-1BB or HER2 or protein domains of 4-1BB or HER2. Fragments of 4-1BB or HER2 as described herein retain the capability of the full-length 4-1BB or HER2 to be recognized and/or bound by a lipocalin mutein, an antibody, and/or a fusion protein of the disclosure.

As used herein, a “variant” of a protein described herein may generally refer to a variant protein having an amino acid sequence which is at least about 60%, 70%, 80%, 90%, 95%, 96%, 97%, 98% or 99% identical to the amino acid sequence of said protein. In some embodiments, the variant may be a naturally occurring variant, such as an alternatively spliced form or naturally-occurring allelic variant of said protein. In some embodiments, the variant is a functional variant.

As used herein, “specific for,” “specific binding,” “specifically bind,” or “binding specificity” relates to the ability of a biomolecule to discriminate between the desired target (for example, 4-1BB or HER2) and one or more reference targets. It is understood that such specificity is not an absolute but a relative property and can be determined, for example, by means of SPR, western blots, ELISA, fluorescence activated cell sorting (FACS), radioimmunoassay (RIA), electrochemiluminescence (ECL), immunoradiometric assay (IRMA), ImmunoHistoChemistry (IHC), and peptide scans. When used herein in the context of the agents described herein or their individual antigen-targeting moiety or moieties, the term “specific for,” “specific binding,” “specifically bind,” or “binding specificity” means that the agents or their antigen-targeting moiety or moieties bind to, react with, or are directed against 4-1BB and/or HER2, as described herein, but do not essentially bind another protein. The term “another protein” includes any proteins that are not 4-1BB or HER2 or proteins closely related to or being homologous to 4-1BB or HER2. However, 4-1BB or HER2 from species other than human and fragments and/or (naturally occurring) variants of 4-1BB or HER2 are not excluded by the term “another protein.” The term “does not essentially bind” means that the agents described herein or their individual antigen-targeting moiety or moieties bind another protein with lower binding affinity than 4-1BB and/or HER2, i.e., show a cross-reactivity of less than 30%, preferably 20%, more preferably 10%, particularly preferably less than 9, 8, 7, 6, or 5%. Whether the agents described herein or their individual antigen-targeting moiety or moieties react as defined herein above can easily be tested, inter alia, by comparing the reaction the agents described herein or their individual antigen-targeting moiety or moieties with 4-1BB and/or HER2 and the reaction of the agents described herein or their individual antigen-targeting moiety or moieties with (an)other protein(s).

The term “small molecule”, as used herein, generally refers to a low molecular weight (e.g., <900 Daltons) organic compound.

As used herein, “bispecific” refers to a molecule is able to specifically bind to at least two distinct targets. Typically, a bispecific molecule comprises two target-binding sites, each of which is specific for a different target. In some embodiments, the bispecific molecule is capable of simultaneously binding two targets.

As used interchangeably herein, the terms “conjugate,” “conjugation,” “fuse,” “fusion,” or “linked” refer to the joining together of two or more subunits, through all forms of covalent or non-covalent linkage, by means including, but not limited to, genetic fusion, chemical conjugation, coupling through a linker or a cross-linking agent, and non-covalent association.

The term “fusion polypeptide” or “fusion protein” as used herein refers to a polypeptide or protein comprising two or more subunits. In some embodiments, a fusion protein as described herein comprises two or more subunits, at least one of these subunits being capable of specifically binding to 4-1BB, and a further subunit capable of specifically binding to a tumor antigen, e.g., a tumor antigen expressed on the surface of a tumor, such as HER2. Within the fusion protein, these subunits may be linked by covalent or non-covalent linkage. Preferably, the fusion protein is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. However, the subunits of a fusion protein of the present disclosure may also be linked through chemical conjugation. The subunits forming the fusion protein are typically linked to each other as follows: C-terminus of one subunit to N-terminus of another subunit, or C-terminus of one subunit to C-terminus of another subunit, or N-terminus of one subunit to N-terminus of another subunit, or N-terminus of one subunit to C-terminus of another subunit. The subunits of the fusion protein can be linked in any order and may include more than one of any of the constituent subunits. If one or more of the subunits is part of a protein (complex) that consists of more than one polypeptide chain, the term “fusion protein” may also refer to the protein comprising the fused sequences and all other polypeptide chain(s) of the protein (complex). As an illustrative example, where a full-length immunoglobulin is fused to a lipocalin mutein via a heavy or light chain of the immunoglobulin, the term “fusion protein” may refer to the single polypeptide chain comprising the lipocalin mutein and the heavy or light chain of the immunoglobulin. The term “fusion protein” may also refer to the entire immunoglobulin (both light and heavy chains) and the lipocalin mutein fused to one or both of its heavy and/or light chains.

As used herein, the term “subunit” of a fusion protein disclosed herein refers to a single protein or a separate polypeptide chain, which may form a stable folded structure by itself and define a unique function of providing a binding motif towards a target. In some embodiments, a preferred subunit of the disclosure is a lipocalin mutein. In some other embodiments, a preferred subunit of the disclosure is a full-length immunoglobulin or an antigen-binding domain thereof.

A “linker” that may be comprised by a fusion protein of the present disclosure joins together two or more subunits of a fusion protein as described herein. The linkage can be covalent or non-covalent. A preferred covalent linkage is via a peptide bond, such as a peptide bond between amino acids. A preferred linker is a peptide linker. Accordingly, in a preferred embodiment, said linker comprises one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. Exemplary linkers include, but are not limited to, the linkers with the amino acid sequences of SEQ ID NOs: 4-14. Other preferred linkers include chemical linkers.

The term “PRS-343”, also known as “cinrebafusp alfa”, refers to the 4-1BB/HER2-bispecific fusion protein having the amino acid sequences of SEQ ID NOs: 50 and 51. The overall structure of PRS-343 is shown in FIG. 13D.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100. A skilled artisan will recognize available computer programs, for example BLAST (Altschul et al., 1997), BLAST2 (Altschul et al., 1990), FASTA (Pearson and Lipman, 1988), GAP (Needleman and Wunsch, 1970), Smith-Waterman (Smith and Waterman, 1981), and Wisconsin GCG Package, for determining sequence identity using standard parameters. The percentage of sequence identity can, for example, be determined herein using the program BLASTP, version 2.2.5, Nov. 16, 2002 (Altschul et al., 1997), calculating the percentage of numbers of “positives” (homologous amino acids) from the total number of amino acids selected for the alignment.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity.

A “sample” is defined as a biological sample taken from any subject. Biological samples include, but are not limited to, blood, serum, urine, feces, semen, or tissue, including tumor tissue. Preferably, the biological sample is blood serum or blood plasma.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, mice, pigs, apes such as cynomolgus monkeys, to name only a few illustrative examples. Preferably, the “mammal” used herein is human. As used herein, the term “patient” refers to a subject as defined above, preferably to a human patient.

As used herein, the term “kit of parts” (in short: “kit”) refers to an article of manufacture comprising one or more containers and, optionally, a data carrier. Said container(s) may be filled with the (re-)agents and compositions as described herein. Additional containers may be included in the kit that contain, e.g., diluents, buffers and/or further (re-)agents or compositions. Said data carrier may be a non-electronic data carrier, e.g., a graphical data carrier such as an information leaflet, an information sheet, a bar code or an access code, or an electronic data carrier, such a CD, a DVD, a microchip or another semiconductor-based electronical data carrier. The access code may allow access to a database. Said data carrier may comprise instructions for using the (re-)agents and compositions as described herein and/or performing the methods and uses described herein.

As used herein the term “about” or “approximately” means within 20%, preferably within 15%, preferably within 10%, and more preferably within 5% of a given value or range. It also includes the concrete number, i.e., “about 20” includes the number of 20. The term “at least about” as used herein includes the concrete number, i.e., “at least about 20” includes 20.

As used herein, the term “and/or” includes the meaning of “and,” “or,” and “all or any other combination of the elements connected by said term”.

As used herein, the singular forms “a,” “an,” and “the” include plural referents unless the context clearly dictates otherwise.

### IV. DESCRIPTIONS OF FIGURES

FIG. 1: provides the results of an in vitro T cell immunogenicity assessment of the HER2/4-1BB bispecific fusion proteins (SEQ ID NOs: 50 and 51, SEQ ID NOs: 50 and 53, SEQ ID NOs: 52 and 49, and SEQ ID NOs: 54 and 49), reference antibody SEQ ID NOs: 50 and 48, and positive control keyhole limpet hemocyanine (KLH). The assay was performed using a PBMC-based format as described in Example 1, with 32 donors and human leukocyte antigen (HLA) allotypes reflective of the distribution in a global population. FIG. 1A presents the stimulation index (proliferation in the presence vs. absence of test article). The average responses are indicated as bars. The threshold that defines a responding donor (stimulation index >2) is indicated as a dotted line. FIG. 2B shows the number of responders for each test article.

FIG. 2: shows the cell-based activity of PRS-343 to co-stimulate T cell activation in a target-dependent manner. Purified human T cells (FIG. 2A) or a 4-1BB overexpressing-Jurkat NF-κB reporter cell line (FIG. 2B) were co-cultured with HER2-expressing tumor cell lines (NCI-N87 (HER2 high), MKN45 (HER2 low), and HepG2 (HER2 null)), orwithout tumor cells, in the presence of PRS-343. In the presence of HER2-positive cell lines, a dose-dependent induction of IL-2 or 4-1BB clustering and downstream signaling in Jurkat NF-κB reporter cells was observed with PRS-343. All data depicted here are representative illustrations of experiments carried out with minimum two different donors. Statistical analysis: *, P<0.05; **, P<0.01; and ***, P<0.001, using one-way ANOVA with Dunnet multiple comparison test.

FIG. 3: depicts the accelerated titration design of the Phase 1, open-label, dose escalation study of PRS-343 (FIG. 3A) and the overall study design (FIG. 3B).

FIG. 4: depicts the overall study design.

FIG. 5: shows the geometric mean PRS-343 serum concentration-time profiles after a single dose (the first dose, administered Cycle 1 Day 1 administration), ranging from 0.015 mg/kg to 8 mg/kg. The 8 mg/kg plot includes patients in both Cohort 11 (8 mg/kg, Q3W) and 11B (8 mg/kg, Q2W).

FIG. 6: presents the drug exposure/pharmacodynamics relationship for Cohorts 1 to 11B (dose levels ranging from 0.0005 mg/kg Q3W to 8 mg/kg Q2W).

FIG. 7: shows the CD8+ T cell expansion in full tumor tissue (FIG. 7A), tumor stroma (FIG. 7B), and tumor cells (FIG. 7C) in patients receiving PRS-343. The increase of CD8+ T cells is more pronounced for patients in Cohort 9 of the study and onwards (dose levels >2.5 mg/kg) as compared to low dose Cohorts 1-8.

FIG. 8: shows the CD8+ T cell expansion in full tumor tissue (FIG. 8A), tumor stroma (FIG. 8B), and tumor cells (FIG. 8C) in the responding patient 107-012. The increase of CD8+ T cells are more pronounced in tumor cells than in full tumor tissue or tumor stroma.

FIG. 9: shows the CD8+ T cell expansion in full tumor tissue (FIG. 9A), tumor stroma (FIG. 9B), and tumor cells (FIG. 9C) in the responding patient 108-002. The increase of CD8+ T cells are more pronounced in tumor cells than in full tumor tissue or tumor stroma.

FIG. 10: shows the CD8+Ki67+ T cell expansion in full tumor tissue (FIG. 10A), tumor stroma (FIG. 10B), and tumor cells (FIG. 10C) in the responding patient 108-002. The increase of CD8+Ki67+ T cells is only observed in tumor cells.

FIG. 11: shows the average time on treatment with PRS-343 is increased in Cohort 11B (8 mg/kg, Q2W) compared to Cohorts 9 to 11 (2.5 mg/kg, 5 mg/kg, and 8 mg/kg, respectively, Q3W).

FIG. 12: depicts the best response in target lesions for Cohorts 1 to 11 B (FIG. 12A) and Cohorts 9 to 11 B (FIG. 12B).

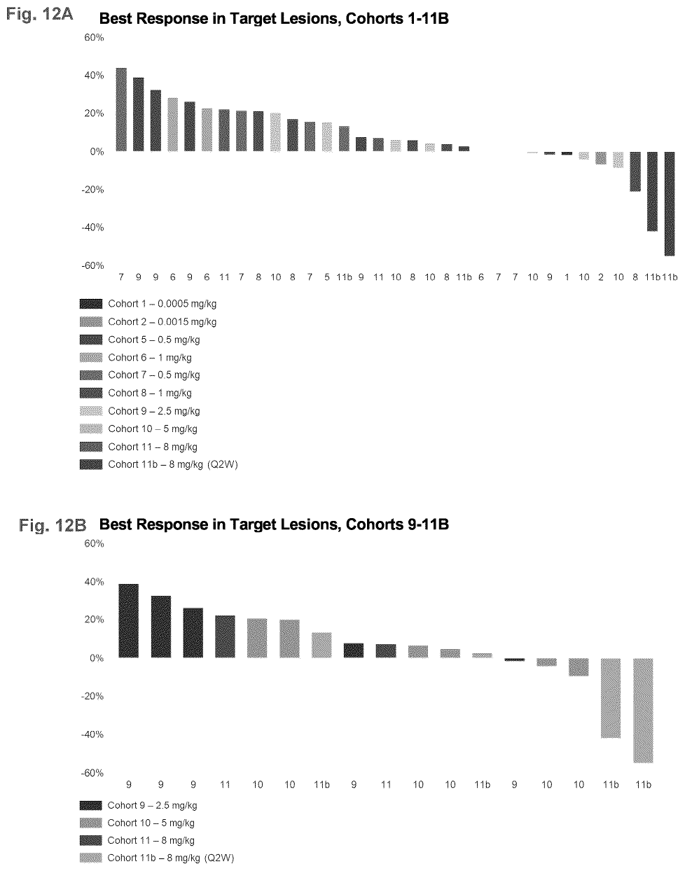

FIG. 13: provides an overview over the design of HER2/4-1BB bispecific fusion proteins as described herein. Representative HER2/4-1BB bispecific fusion proteins were made based on an antibody specific for HER2 (e.g., an antibody shown in SEQ ID NOs: 50 and 48) and a lipocalin muteins specific for 4-1BB (e.g., a lipocalin mutein shown in SEQ ID NO: 22). One or more anti-4-1BB lipocalin muteins were genetically fused, via a peptide linker, at the N-terminus or the C-terminus, to an anti-HER2 antibody at the C-terminus of the antibody heavy chain domain (HC) (FIG. 13D), the N-terminus of the HC (FIG. 13A), the C-terminus of the antibody light chain (LC) (FIG. 13C), and/or the N-terminus of the LC (FIG. 13B), resulting in the fusion proteins such as SEQ ID NOs: 50 and 51, SEQ ID NOs: 50 and 53, SEQ ID NOs: 52 and 49, and SEQ ID NOs: 54 and 49. An engineered IgG4 backbone with the mutations S228P, F234A, and L235A was used for the anti-HER2 antibody as included in the fusion proteins.

FIG. 14: shows the geometric mean PRS-343 serum concentration-time profiles after a single dose (the first dose, cycle 1, day 1), ranging from 0.015 mg/kg to 18 mg/kg. The 8 mg/kg plot includes patients in both Cohort 11 (8 mg/kg, Q3W) and 11B (8 mg/kg, Q2WA). The 12 mg/kg plot includes patients in Cohort 12B (12 mg/kg, Q2WA), the 18 mg/kg includes patients in Cohort 13B (18 mg/kg, Q2WA).

FIG. 15: shows CD8+ T cell expansion in full tumor tissue (FIG. 15A) and serum levels of soluble 4-1BB (s4-1BB) (FIG. 15 B) of patients in non-active dose Cohorts 1-8 vs. patients in the active dose Cohorts 9-13B. Patients treated with an active dose of PRS-343 showed increased CD8+ T cells in the tumor tissue and circulating s4-1 BB, demonstrating 4-1 BB arm activity of PRS-343.

FIG. 16: shows the course of treatment for patients in Cohorts 11B, 11C, 12B, 13B and Obi+11B, including the clinical status (where applicable).

FIG. 17: depicts the best response in target lesions for Cohorts 9, 10, 11, 11B, 11C, 12B, 13B and Obi+11B.

FIG. 18: shows CD8+ T cell expansion (x-fold induction) vs. % growth/shrinkage of target lesion in active dose cohorts. Patients with SD≥C6, PR and CR exhibited an at least 2.3-fold increase of CD8+ T cells.

FIG. 19: shows CT scans of a target lesion (lung; see dark circle) in the responding patient 103-021 at baseline, C2 post-treatment and C6 post-treatment. The patient showed a complete response (CR).

FIG. 20: shows post-treatment CD8+ T cell expansion in full tumor tissue (FIG. 20A) and an increase of circulating s4-1BB in the serum (FIG. 20B) of the CR patient 103-021, demonstrating 4-1BB arm activity of PRS-343.

FIG. 21: shows CT scans of target lesions (see dark circles) in the responding patient 107-012 at baseline and C4 post-treatment. The patient showed a partial response (PR).

FIG. 22: shows post-treatment CD8+ T cell and CD8+Ki67+ T cell expansion in full tumor tissue (FIG. 22A) and an increase of circulating s4-1BB in the serum (FIG. 22B) of the PR patient 107-012, demonstrating 4-1BB arm activity of PRS-343.

FIG. 23: shows a repeated increase of circulating s4-1BB in the serum of the PR patient 103-012 over the course of multiple treatment cycles.

FIG. 24: shows pre-treatment absolute numbers of CD8+ T cells in full tumor tissue of active cohort patients split up in “PD & SD<C6” and “CR, PR & SD>C6” patients (FIG. 24A) and a plot of % PD-L1+ cells of total immune cells (IC score) vs. pre-treatment absolute numbers of CD8+ T cells for individual responding patients of active dose cohorts (FIG. 24B). PRS-343 drives clinical benefit in PD-L1 low/negative patients and patients with low CD8+ T cell counts prior to therapy.

FIG. 25: shows dose dependency of serum levels of s4-1BB (measured over the course of cycle 1) upon treatment with PRS-343 across all tested dose cohorts (8 mg/kg data include data of patients treated Q1W, Q2W or Q3W). The drop of s4-1BB serum levels at the 18 mg/kg dose may indicate overactivation of the 4-1BB pathway or the potential for overactivation of the 4-1BB pathway. Baseline: s4-1BB serum levels before treatment with PRS-343; grey line: connects group averages; black lines: median; Mann-Whitney U test was used for statistical analysis.

FIG. 26: shows the maximum fold-induction of s4-1BB levels in the serum of five HER2 low patients (HER2 IHC2+/FISH- or IHC1+/FISH-) upon treatment with PRS-343. The maximum fold-induction of s4-1BB in cycle 1 was significantly higher in patients with a clinical response (stable disease, SD) than in patients with progressive disease (PD). Baseline: s4-1BB serum levels before treatment with PRS-343.

FIG. 27: shows the s4-1BB profiles of the two HER2 low patients with clinical benefit, breast cancer patient 103-016 (FIG. 27A; stable disease at cycles 2 and 4) and colorectal cancer patient 103-019 (FIG. 27B; stable disease at cycles 2, 4 and 6). Biopsy analysis revealed that the tumors of these patients were characterized by a low expression of HER2.

### V. DETAILED DESCRIPTION OF THE DISCLOSURE

The present disclosure relies in part on the results of a first-in-human Phase 1 study of PRS-343 conducted in patients with (presumed) HER2+ advanced or metastatic solid tumors to assess the safety and efficacy of PRS-343, as also described in WO 2021/089588 A1, which is incorporated herein by reference in its entirety.

In one aspect, the present disclosure provides a method of predicting a positive clinical outcome for a cancer patient upon treatment with a 4-1BB agonistic agent, said method comprising (a) measuring the level of soluble 4-1BB (s4-1BB) in a biological sample obtained from the cancer patient prior to administering the 4-1BB agonistic agent to the cancer patient; and (b) measuring the level of s4-1BB in a biological sample obtained from the cancer patient after administering the 4-1BB agonistic agent to the cancer patient, wherein a positive clinical outcome is predicted if the level of s4-1BB in the biological sample obtained from the cancer patient after administering the 4-1BB agonistic agent to the cancer patient is increased as compared to the level of s4-1BB in the biological sample obtained from the cancer patient prior to administering the 4-1BB agonistic agent to the cancer patient.

In some embodiments, the positive clinical outcome comprises stable disease (SD), partial response (PR), complete response (CR), increased overall survival (OS) and/or increased progression free survival (PFS).

In another aspect, the present disclosure provides a method of assessing activity of a 4-1BB agonistic agent in a subject, preferably a cancer patient, treated with the 4-1BB agonistic agent, said method comprising (a) measuring the level of s4-1BB in a biological sample obtained from the subject prior to administering the 4-1BB agonistic agent to the subject; and (b) measuring the level of s4-1BB in a biological sample obtained from the subject after administering the 4-1BB agonistic agent to the subject, wherein a level of s4-1BB in the biological sample obtained from the subject after administering the 4-1BB agonistic agent to the subject which is increased as compared to the level of s4-1BB in the biological sample obtained from the subject prior to administering the 4-1BB agonistic agent to the subject indicates activity of the 4-1BB agonistic agent in the subject.

In some embodiments, the activity is dose-dependent activity.

In some embodiments, the activity is activation of 4-1BB signaling.

In some embodiments, the activity of the 4-1BB agonistic agent is assessed in a plurality of subjects.

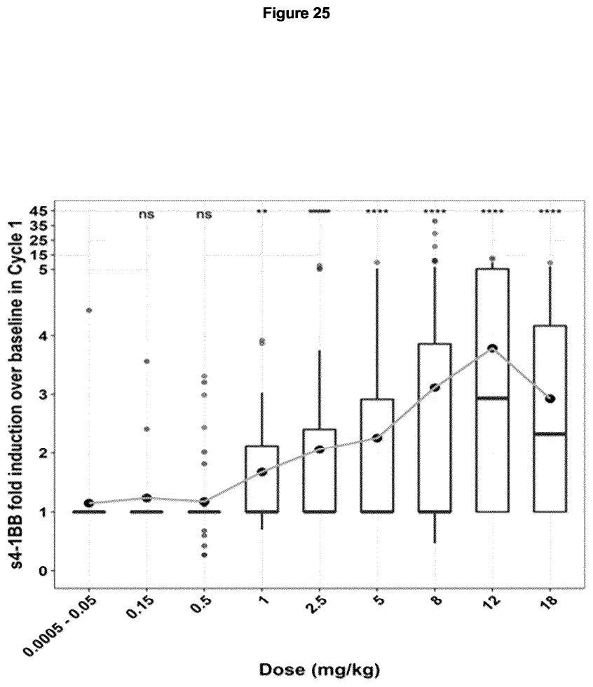

In some embodiments, the method further comprises generating a dose response curve, e.g., a dose response curve as shown in FIG. 25.

In another aspect, the present disclosure provides a method of selecting a dose of a 4-1BB agonistic agent for treating a disease, e.g., cancer, said method comprising assessing activity of the 4-1BB agonistic agent in accordance with the method as defined above.

In another aspect, the present disclosure provides a method of selecting a dose of a 4-1BB agonistic agent for treating a disease, e.g., cancer, said method comprising (a) measuring the level of s4-1BB in biological samples obtained from a plurality of subjects having the disease upon administration of different doses of the 4-1BB agonistic agent, and (b) generating a dose response curve based on the results obtained in step (a), wherein, if the level (e.g., an average level) of s4-1BB decreases at a dose X, a dose which is lower than dose X is selected as the dose for treating the disease.

In some embodiments, the decrease of the level of s4-1BB at dose X indicates an overactivation of the 4-1BB pathway or a potential for overactivation of the 4-1BB pathway.

In some embodiments, the dose is a maintenance dose which is administered after administration of an initial higher dose (i.e., loading dose).

In another aspect, the present disclosure provides a method of treating a cancer patient comprising administering an effective amount of a 4-1BB agonistic agent to the cancer patient, said method comprising the steps: (a) measuring the level of s4-1BB in a biological sample obtained from the cancer patient prior to administering the 4-1BB agonistic agent to the cancer patient; (b) administering the 4-1BB agonistic agent to the cancer patient; (c) measuring the level of s4-1BB in a biological sample obtained from the cancer patient after administering the 4-1BB agonistic agent to the cancer patient; and (d) continuing to administer the 4-1BB agonistic agent to the cancer patient, if the level of s4-1BB in the biological sample obtained from the cancer patient after administering the 4-1BB agonistic agent to the cancer patient is increased as compared to the level of s4-1BB in the biological sample obtained from the cancer patient prior to administering the 4-1BB agonistic agent to the cancer patient.

In some embodiments, administration of the 4-1BB agonistic agent to the cancer patient is discontinued if the level of s4-1BB in the biological sample obtained from the cancer patient after administering the 4-1BB agonistic agent to the cancer patient is not increased as compared to the level of s4-1BB in the biological sample obtained from the cancer patient prior to administering the 4-1BB agonistic agent to the cancer patient.

In some embodiments of the above methods, the biological sample is blood serum or blood plasma.

In some embodiments, the level of s4-1BB in the biological sample obtained from the subject/cancer patient after administering the 4-1BB agonistic agent is increased by at least about 1.1-fold, at least about 1.2-fold, at least about 1.3-fold, at least about 1.4-fold, at least about 1.5-fold, at least about 2-fold, at least about 2.5-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold or even more fold, as compared to the level of s4-1BB in the biological sample obtained from the subject/cancer patient prior to administering the 4-1BB agonistic agent to the subject/cancer patient.

In some embodiments, the level of s4-1BB in the biological sample obtained from the subject/cancer patient after administering the 4-1BB agonistic agent is increased by about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml of the biological sample, as compared to the level of s4-1BB in the biological sample obtained from the subject/cancer patient prior to administering the 4-1BB agonistic agent to the subject/cancer patient.

In some embodiments, the level of s4-1BB in the biological sample obtained from the subject/cancer patient after administering the 4-1BB agonistic agent is increased to a concentration of about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml of the biological sample.

In some embodiments, measuring the level of s4-1BB in a biological sample obtained from the subject/cancer patient after administering the 4-1BB agonistic agent to the subject/cancer patient comprises measuring the levels of s4-1BB during and/or after multiple (e.g., two, three, four, or more) cycles of treatment with the 4-1BB agonistic agent.

In some embodiments, the level of s4-1BB in the biological sample obtained from the subject/cancer patient after administering the 4-1BB agonistic agent to the subject/cancer patient is increased as compared to the level of s4-1BB in the biological sample obtained from the subject/cancer patient prior to administering the 4-1BB agonistic agent to the subject/cancer patient during or after at least one, at least two, at least three, or at least four cycle(s) of treatment with the 4-1BB agonistic agent.

In some embodiments, the level of s4-1BB in the biological sample obtained from the subject/cancer patient after administering the 4-1BB agonistic agent to the subject/cancer patient is repeatedly increased as compared to the level of s4-1BB in the biological sample obtained from the subject/cancer patient prior to administering the 4-1BB agonistic agent to the subject/cancer patient during or after multiple (e.g., two, three, four, or more) cycles of treatment with the 4-1BB agonistic agent.

In some embodiments, the level of s4-1BB in the biological sample obtained from the subject/cancer patient after administering the 4-1BB agonistic agent to the subject/cancer patient is increased as compared to the level of s4-1BB in the biological sample obtained from the subject/cancer patient prior to administering the 4-1BB agonistic agent to the subject/cancer patient during or after each of the multiple (e.g., two, three, four, or more) cycles of treatment with the 4-1BB agonistic agent.

In some embodiments, the cycle of treatment with the 4-1BB agonistic agent comprises: (i) about 21 days, wherein the 4-1BB agonistic agent is administered at an interval of about once every three weeks (Q3W); (ii) about 28 days, wherein the 4-1BB agonistic agent is administered at an interval of about once every two weeks (Q2W); or (iii) about 21 days, wherein the 4-1BB agonistic agent is administered at an interval of about once every week (Q1W).

In some embodiments, the level of s4-1BB in the biological sample obtained from the subject/cancer patient after administering the 4-1BB agonistic agent to the subject/cancer patient is the maximum level of s4-1BB measured during and/or after one or more (e.g., two, three, four, or more) cycles of treatment with the 4-1BB agonistic agent. In some embodiments, it is the maximum level of s4-1BB measured during the first cycle of treatment with the 4-1BB agonistic agent.

In some embodiments, the maximum level of s4-1BB measured during and/or after one or more (e.g., two, three, four, or more) cycles of treatment with the 4-1BB agonistic agent is increased by at least about 15, at least about 20, at least about 25, at least about 30, at least about 35, at least about 40 or even more folds, as compared to the level of s4-1BB in the biological sample obtained from the subject/cancer patient prior to administering the 4-1BB agonistic agent to the subject/cancer patient.

In some embodiments, the level of s4-1BB in the biological sample obtained from the subject/cancer patient after administering the 4-1BB agonistic agent to the subject/cancer patient is the average level of s4-1BB measured during and/or after one or more (e.g., two, three, four, or more) cycles of treatment with the 4-1BB agonistic agent.

In another aspect, the present disclosure provides the use of s4-1BB as a predictive biomarker (e.g., serum-based biomarker) for the clinical outcome of a cancer patient upon treatment with a 4-1BB agonistic agent.

In another aspect, the present disclosure provides the use of s4-1BB as a biomarker (e.g., serum-based biomarker) for activity, preferably dose-dependent activity, of a 4-1BB agonistic agent in a subject, preferably a cancer patient, treated with the 4-1BB agonistic agent.

In another aspect, the present disclosure provides the use of s4-1BB as a biomarker (e.g., serum-based biomarker) for selecting a dose (e.g., maintenance dose) of a 4-1BB agonistic agent for treating a disease, e.g., cancer.

In another aspect, the present disclosure provides the use of a kit comprising means for detecting s4-1BB in a biological sample for predicting a positive clinical outcome for a cancer patient upon treatment with a 4-1BB agonistic agent.

In another aspect, the present disclosure provides the use of a kit comprising means for detecting s4-1BB in a biological sample for assessing activity, preferably dose-dependent activity, of a 4-1BB agonistic agent in a subject, preferably a cancer patient, treated with the 4-1BB agonistic agent.

In another aspect, the present disclosure provides the use of a kit comprising means for detecting s4-1BB in a biological sample for selecting a dose (e.g., maintenance dose) of a 4-1BB agonistic agent for treating a disease, e.g., cancer.

In some embodiments of the above uses, the biological sample is blood serum or blood plasma.

In some embodiments, the means for detecting s4-1BB in a biological sample comprise an antibody specific for 4-1BB and/or s4-1BB.

In some embodiments, the kit is an immunoassay kit.

In some embodiments, the kit further comprises one or more of the following: a container containing a diluent, a container containing a buffer, a container containing an enzyme-conjugate, a container containing a substrate solution, a container containing a secondary antibody, a container containing beads, a multi-well plate, a data carrier.

In some embodiments, the 4-1BB agonistic agent is selected from the group consisting of antibodies and antigen-binding fragments thereof, antibody mimetics, small molecules and other antigen-binding molecules, such as aptamers, having 4-1BB agonistic activity. In some embodiments, the antibody mimetics are selected from the group consisting of Affibody molecules, Affilins, Affimers, Affitins, Alphabodies, lipocalin muteins, Avimers, DARPins, Fynomers, Kunitz domain peptides, Monobodies and nanoCLAMPs. In some embodiments, the 4-1BB agonistic agent is a bi-specific agent.

In some embodiments of the above methods or uses, the 4-1BB agonistic agent comprises or is a lipocalin mutein specific for 4-1BB.

In some embodiments, the lipocalin mutein is a mutein of mature human neutrophil gelatinase-associated lipocalin (hNGAL) having binding specificity for 4-1BB. A mutein of mature hNGAL may be designated herein as an “hNGAL mutein”.

In some embodiments, the lipocalin mutein is capable of binding human 4-1BB with high affinity and/or co-stimulating human T cells when immobilized on a plastic dish together with an anti-CD3 antibody. In some embodiments, the lipocalin mutein comprises an amino acid sequence selected from the group consisting of SEQ ID NOs: 21-39 or of a fragment or variant thereof. In some embodiments, the lipocalin mutein has the amino acid sequence shown in SEQ ID NO: 22. In some embodiments, the lipocalin mutein has an amino acid sequence with high sequence identity, such as at least 70%, at least 75%, at least 80%, at least 82%, at least 85%, at least 87%, at least 90%, at least 95%, at least 98%, at least 99%, or higher identity, to an amino acid sequence selected from the group consisting of SEQ ID NOs: 21-39. In some embodiments the lipocalin mutein has an amino acid sequence with high sequence identity, such as at least 70%, at least 75%, at least 80%, at least 82%, at least 85%, at least 87%, at least 90%, at least 95%, at least 98%, at least 99%, or higher identity, to the amino acid sequence shown in SEQ ID NOs: 22. Suitable lipocalin muteins that are specific for 4-1BB are also described in WO 2016/177762 A1, which is incorporated herein by reference in its entirety.

In some embodiments, the lipocalin mutein comprises the amino acid sequence shown in SEQ ID NO: 22 or an amino acid sequence having at least 95% sequence identity to the amino acid sequence shown in SEQ ID NO: 22.

In some embodiments, the 4-1BB agonistic agent is part of a fusion molecule, in particular a fusion protein, comprising the 4-1BB agonistic agent and a tumor-targeting moiety. The tumor-targeting moiety may be selected from the group consisting of antibodies and antigen-binding fragments thereof, antibody mimetics, small molecules and other antigen-binding molecules, such as aptamers. In some embodiments, the antibody mimetics are selected from the group consisting of Affibody molecules, Affilins, Affimers, Affitins, Alphabodies, lipocalin muteins, Avimers, DARPins, Fynomers, Kunitz domain peptides, Monobodies and nanoCLAMPs. In some embodiments, the tumor-targeting moiety is specific for a tumor antigen expressed on the surface of a tumor cell. In some embodiments, the tumor-targeting moiety comprises an antibody or an antigen-binding fragment thereof. In some embodiments, the fusion molecule is a fusion protein comprising an antibody specific for a tumor antigen expressed on the surface of a tumor cell and a lipocalin mutein specific for 4-1BB, e.g., a lipocalin mutein as defined above, wherein, preferably, the antibody is fused at the C-terminus of both heavy chains to the N-terminus of a lipocalin mutein specific for 4-1BB.

In some embodiments, the tumor antigen is HER2. In some embodiments, the 4-1BB agonistic agent is a 4-1BB/HER2-bispecific agent.

In some embodiments, the 4-1BB/HER2-bispecific agent comprises at least one 4-1BB-targeting moiety having 4-1BB agonistic activity and at least one HER2-targeting moiety, wherein the targeting moieties are independently selected from the group consisting of antibodies and antigen-binding fragments thereof, antibody mimetics, small molecules and other antigen-binding molecules, such as aptamers. In some embodiments, the antibody mimetics are selected from the group consisting of Affibody molecules, Affilins, Affimers, Affitins, Alphabodies, lipocalin muteins, Avimers, DARPins, Fynomers, Kunitz domain peptides, Monobodies and nanoCLAMPs. HER2-targeting antibodies are known in the art and include, for example, trastuzumab and pertuzumab. 4-1BB-targeting antibodies are also known in the art and include, for example, urelumab and utomilumab. In some embodiments, the 4-1BB/HER2-bispecific agent is a conjugate or a fusion molecule, in particular a fusion protein. In some embodiments, the 4-1BB/HER2-bispecific agent is a bispecific antibody.

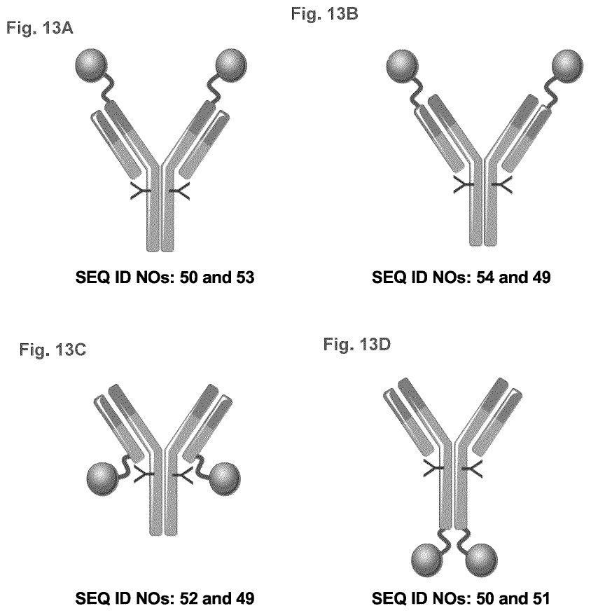

In some embodiments, the 4-1BB/HER2-bispecific agent is a fusion molecule, particularly a fusion protein comprising an antibody or an antigen-binding domain thereof specific for HER2 and at least one lipocalin mutein specific for 4-1BB, e.g., a lipocalin mutein as defined above. More particularly, the fusion protein may comprise at least two subunits in any order: (1) a first subunit that comprises an antibody or an antigen-binding domain thereof specific for HER2, and (2) a second subunit that comprises a lipocalin mutein specific for 4-1BB. In some embodiments, the fusion protein contains at least one additional subunit, for example, a third subunit. In some embodiments, the fusion protein contains a third subunit that comprises a lipocalin mutein specific for 4-1BB. In some embodiments, at least one subunit of the fusion protein is fused at its N-terminus and/or its C-terminus to another subunit. In some embodiments, at least one subunit of the fusion protein is fused to another subunit via a linker. A linker as described herein may be a peptide linker, for example, an unstructured glycine-serine (GS) linker, a glycosylated GS linker, or a proline-alanine-serine polymer (PAS) linker. In some embodiments, a (Gly₄Ser)₃ linker ((G₄S)₃) as shown in SEQ ID NO: 4 is used. Other exemplary linkers are shown in SEQ ID NOs: 5-14. In some embodiments, the second subunit of the fusion protein is linked via a linker, preferably a (G₄S)₃ linker, at its N-terminus to each of the C-terminus of the heavy chain constant region (CH) of the antibody or an antigen-binding domain thereof comprised in the first subunit. In some embodiments, a lipocalin mutein subunit is fused to an antibody subunit of the fusion protein via a peptide linker. In some embodiments, a lipocalin mutein subunit is fused, via a peptide linker, at its N-terminus or its C-terminus to an antibody subunit at the C-terminus of the antibody heavy chain (HC), the N-terminus of the HC, the C-terminus of the antibody light chain (LC), and/or the N-terminus of the LC (e.g., as shown in FIG. 13). In some particular embodiments, the 4-1BB/HER2-bispecific agent is a fusion protein comprising an antibody specific for HER2 fused at the C-terminus of both heavy chains to the N-terminus of a lipocalin mutein specific for 4-1BB, preferably via a peptide linker, e.g., a (G₄S)₃ linker.

In some embodiments, the Fc function of the Fc region of the antibody or an antigen-binding domain thereof comprised in the fusion protein is preserved. Accordingly, the fusion protein may be capable of binding Fc receptor-positive cell at the same time while simultaneously engaging 4-1BB and HER2. In some other embodiments, the Fc function of the Fc region of the antibody or an antigen-binding domain thereof comprised in the fusion protein is reduced or fully suppressed, while the fusion protein is simultaneously engaging 4-1BB and HER2. In some embodiments, this may be achieved, for example, by switching from the IgG1 backbone to IgG4, as IgG4 is known to display reduced Fc-gamma receptor interactions compared to IgG1. In some embodiments, to further reduce the residual binding to Fc-gamma receptors, mutations may be introduced into the IgG4 backbone such as F234A and L235A. In some embodiments, an S228P mutation may also be introduced into the IgG4 backbone to minimize the exchange of IgG4 half-antibody (Silva et al., 2015). In some embodiments, F234A and L235A mutations may be introduced for decreased ADCC and ADCP (Glaesner et al., 2010) and/or M428L and N434S mutations or M252Y, S254T, and T256E mutations for extended serum half-life (Dall′Acqua et al., 2006, Zalevsky et al., 2010). In some embodiments, an additional N297A mutation may be present in the antibody heavy chain of the fusion protein in order to remove the natural glycosylation motif.

In some embodiments, the antibody or antigen-binding domain thereof comprised in the fusion protein comprises the three heavy chain complementarity-determining regions (CDRs) shown in SEQ ID NO: 40, SEQ ID NO: 41, and SEQ ID NO: 42, and/or the three light chain CDRs shown in SEQ ID NO: 43, SEQ ID NO: 44, and SEQ ID NO: 45. In some embodiments, the antibody or antigen-binding domain thereof comprised in the fusion protein comprises a heavy chain variable region (HCVR) shown in SEQ ID NO: 46, and/or a light chain variable region (LCVR) shown in SEQ ID NO: 47. In some embodiments, the antibody or antigen-binding domain thereof comprised in the fusion protein comprises a heavy chain shown in SEQ ID NO: 49, and/or a light chain shown in SEQ ID NO: 50. In some embodiments, the antibody or antigen-binding domain thereof comprised in the fusion protein has a HCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 46, and/or a LCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 47. In other embodiments, the antibody or antigen-binding domain thereof comprised in the fusion protein has a heavy chain with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 49, and/or a light chain with at least 70%, at least 75%, at least 80%, at least 85%, at least 90%, at least 92%, at least 95%, at least 97%, at least 98%, at least 99%, or even higher sequence identity to the amino acid sequence shown in SEQ ID NO: 50.

In some embodiments, the antibody comprises: (a) three heavy chain complementarity-determining regions (CDRs) shown in SEQ ID NO: 40, SEQ ID NO: 41, and SEQ ID NO: 42, and three light chain CDRs shown in SEQ ID NO: 43, SEQ ID NO: 44, and SEQ ID NO: 45; and (b) a heavy chain with at least 95% sequence identity to the amino acid sequence shown in SEQ ID NO: 49, and a light chain with at least 95% sequence identity to the amino acid sequence shown in SEQ ID NO: 50.

In some embodiments, the antibody or antigen-binding domain thereof comprised in the fusion protein is an anti-HER2 antibody. In some embodiments, the antibody or antigen-binding domain thereof comprised in the fusion protein is trastuzumab. In some embodiments, the antibody or antigen-binding domain thereof comprised in the fusion protein is trastuzumab with an IgG4 backbone.

In some embodiments, the fusion protein is generated by genetic fusion of a 4-1BB-specific hNGAL mutein to a trastuzumab IgG4 variant, joined by a flexible, non-immunogenic peptide linker.

In some embodiments, the fusion protein comprises the sets of amino acid sequences selected from the group consisting of SEQ ID NOs: 50 and 51, SEQ ID NOs: 50 and 53, SEQ ID NOs: 52 and 49, and SEQ ID NOs: 54 and 49. In some embodiments, the fusion protein comprises amino acid sequences having at least 70%, at least 75%, at least 80%, at least 85%, at least 90%, at least 92%, at least 95%, at least 97%, at least 98%, at least 99%, or higher sequence identity to the amino acid sequences shown in SEQ ID NOs: 50 and 51, SEQ ID NOs: 50 and 53, SEQ ID NOs: 52 and 49, and SEQ ID NOs: 54 and 49. In some embodiments, where the fusion protein comprises more than one amino acid chain, a given value for the sequence identity relates to the average sequence identity normalized by the number of amino acid residues in both amino acid chains. For example, if a fusion protein consists of amino acid chain A having 100 amino acids and amino acid chain B having 50 amino acids, and another fusion protein consists of amino acid chain A′ having 100 amino acids 80% sequence identity to amino acid chain A and amino acid chain B′ having 50 amino acids and 95% sequence identity to amino acid chain B′, the average sequence identity between both fusion proteins will be (100/(100+50))×80%+(50/(100+50))×95%=85% sequence identity. In some preferred embodiments, where a fusion protein comprises more than one amino acid chain, a given value for the sequence identity means that a protein of interest comprises an amino acid sequence that has at least the given value of sequence identity to one chain of the bispecific fusion protein and comprises an amino acid sequence that has at least the given value of sequence identity to the other chain of the fusion protein.

In some embodiments, the fusion protein comprises the amino acid sequences shown in SEQ ID NOs: 50 and 51. In some embodiments, the fusion protein comprises two chains having the amino acid sequence shown in SEQ ID NO: 50 and two chains having the amino acid sequence shown in SEQ ID NO: 51. Suitable 4-1BB/HER2-bispecifc fusion proteins comprising an antibody or an antigen-binding domain thereof specific for HER2 and a lipocalin mutein specific for 4-1BB are also described in WO 2016/177802 A1, which is incorporated herein by reference in its entirety.

In some embodiments, the 4-1BB/HER2-bispecific agent is capable of engaging HER2 and 4-1BB simultaneously. In some embodiments, the 4-1BB/HER2-bispecific agent is capable of inducing 4-1BB clustering and signaling in a HER2-dependent manner. In some embodiments, the 4-1BB/HER2-bispecific agent is capable of activating 4-1BB signaling in a HER2-expressing tumor microenvironment. In some embodiments, the 4-1BB/HER2-bispecific agent is capable of co-stimulating T cell responses and/or enhancing T cell functions in a HER2-expressing tumor microenvironment.

In some embodiments, the 4-1BB/HER2-bispecific agent is administered at an interval of about once every three weeks, about once every two weeks, or about once every week. In some embodiments, the 4-1BB/HER2-bispecific agent is administered at an interval of about once every two weeks. In some embodiments, the 4-1BB/HER2-bispecific agent is administered at a dose of from about 2.5 mg/kg to about 27 mg/kg. In some embodiments, the 4-1BB/HER2 bispecific agent is administered at a dose of about 2.5 mg/kg, about 5 mg/kg, about 8 mg/kg, about 12 mg/kg or about 18 mg/kg. In some embodiments, the 4-1BB/HER2-bispecific agent is administered at a dose of about 8 mg/kg. In some embodiments, the 4-1BB/HER2 bispecific agent is administered at a dose of about 18 mg/kg. In some embodiments, the 4-1BB/HER2-bispecific agent is administered intravenously, e.g., by intravenous infusion. Methods of treating tumors which comprise administering the specific 4-1BB/HER2-bispecific agent cinrebafusp alfa are described in WO 2021/089588 A1, which is incorporated herein by reference in its entirety

In some embodiments of the above methods and uses, the tumor/cancer is a HER2-expressing tumor/cancer.

In some embodiments, the tumor/cancer is characterized by a low expression of HER2. In some embodiments, the tumor/cancer is characterized by a HER2 status of IHC1+ or IHC2+/(F)ISH−. In some embodiments, the tumor/cancer does not exhibit HER2 gene amplification, e.g., as determined by (F)ISH or next generation sequencing (NGS) analysis.

In some embodiments, the tumor/cancer is a HER2-positive (HER2+) tumor/cancer. In some embodiments, the tumor/cancer is characterized by a HER2 status of IHC3+, IHC2+/(F)ISH+ or (F)ISH+, preferably IHC3+ or IHC2+/(F)ISH+. In some embodiments, the tumor/cancer exhibits HER2 gene amplification, e.g., as determined by (F)ISH or next generation sequencing (NGS) analysis.

In some embodiments, the tumor/cancer is selected from the group consisting of gastric cancer, gynecological cancer (e.g., fallopian tube cancer, endometrial cancer or ovarian cancer), breast cancer, lung cancer, in particular non-small cell lung cancer, gallbladder cancer, cholangiocarcinoma, melanoma, esophageal cancer, gastroesophageal cancer (e.g., gastroesophageal junction cancer), colorectal cancer, rectal cancer, colon cancer, pancreatic cancer, biliary tract cancer, salivary duct cancer, bladder cancer, and cancer of unknown primary.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art and that such modifications and variations are considered to be within the scope of this disclosure.

### VI. EXAMPLES

### Example 1: T cell immunogenicity assessment of HER2/4-1BB bispecific fusion proteins

To investigate the risk of the formation of anti-drug antibodies in man, an in-vitro T cell immunogenicity assessment was performed for the HER2/4-1BB bispecific fusion proteins SEQ ID NOs: 50 and 51, SEQ ID NOs: 50 and 53, SEQ ID NOs: 52 and 49 and SEQ ID NOs: 54 and 49, as well as for reference antibody SEQ ID NOs: 50 and 48.

Human peripheral blood mononuclear cells (PBMCs) from 32 donors, selected to cover human leukocyte antigen (HLA) allotypes and reflective of the distribution in a global population, were thawed, washed, and seeded onto 96-well plates at a density of 3×10⁵ cells per well. Test articles, diluted in assay media, were added to the cells at a concentration of 30pg/mL and then incubated for 7 days in a humidified atmosphere at 37° C. and 5% CO₂. Assay medium alone was used as a blank, and keyhole limpet hemocyanine (KLH) was tested as a naive positive control. On day 7, PBMCs were labelled for surface phenotypic CD3+ and CD4+ markers and for DNA-incorporated EdU (5-ethynyl-2′deoxyuridine), used as a cell proliferation marker. The percentage of CD3+CD4+EdU+ proliferating cells was measured using a Guava easyCyte 8HT flow cytometer and analyzed using GuavaSoft InCyte software.

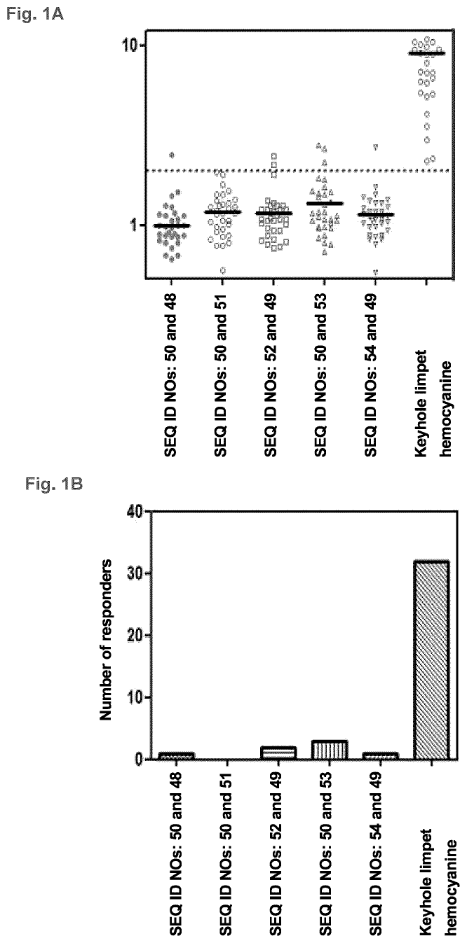

Results of this assay are shown in FIG. 1. In FIG. 1A, the stimulation index was plotted, which was obtained by the ratio of proliferation in the presence vs. absence of test article. The threshold that defines a responding donor (stimulation index>2) is indicated as a dotted line. In FIG. 1B, the number of responding donors as defined by this threshold was plotted. Evidently, the number of donors responding to the reference antibody SEQ ID NOs: 50 and 48 lies at one and is therefore small, while all 32 donors respond to the positive control KLH with strong proliferation above the threshold. For the bispecific fusion proteins, the number of responding donors are zero, one, two, and three for SEQ ID NOs: 50 and 51, SEQ ID NOs: 54 and 49, SEQ ID NOs: 50 and 53, and SEQ ID NOs: 52 and 49, respectively.

The results demonstrate that the bispecific fusion proteins, in particular SEQ ID NOs: 50 and 51 and SEQ ID NOs: 54 and 49, induce little response in the in-vitro T cell immunogenicity assessment, indicating low risk of inducing immunogenic responses in man.

### Example 2: In-vitro T cell activation of PRS-343

HER2 target-dependent T cell activation mediated by PRS-343 was assessed in co-culture experiments using a panel of cell lines expressing different levels of HER2. Cancer cell lines representing a range of clinically relevant levels of HER2 receptor (NCI-N87: HER2 high, MKN45: HER2 low, HepG2: HER2 null) were tested for their ability to mediate clustering of PRS-343 and subsequent activation of T cells. To evaluate a potential therapeutic window, cell lines derived from healthy tissues known to express background levels of HER2 were also included.

Briefly, cancer cells or cells derived from healthy tissue pretreated with 10 pg/mL of mitomycin C (Sigma Aldrich) were seeded in culture plates pre-coated with anti-CD3 and incubated overnight at 37° C. in a humidified 5% CO₂ atmosphere. T cell suspension (5×10⁴ cells) together with test article was added and incubated for 3 days. The level of T cell activation was measured by quantifying of human IL-2 in the supernatant, using an electrochemiluminescence (ECL) immunoassay (using IL2 DuoSet kit; R&D Systems).

Specific activation of the 4-1BB pathway by PRS-343 was also assessed using a luciferase reporter cell assay (Promega), where a 4-1BB overexpressing reporter cell line (NF-κB-Luc2/4-1BB Jurkat cells) was cocultured with HER2-positive tumor cell lines and where 4-1BB pathway activation was measured by luminescence.

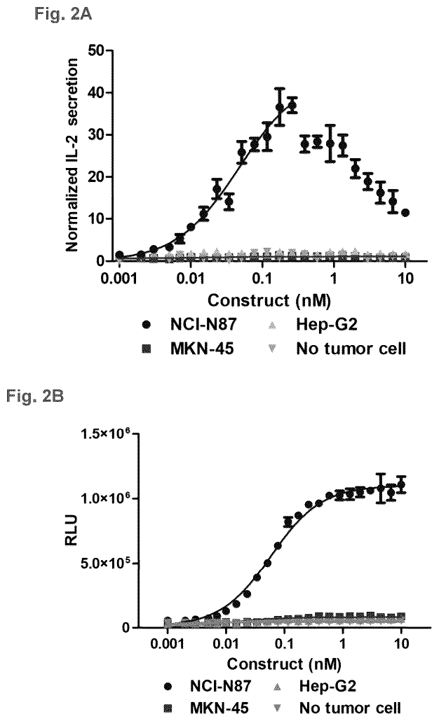

Results of an exemplary experiments are shown in FIG. 2. In the presence of HER2-positive cell lines, a dose-dependent induction of IL-2 was observed with PRS-343. Particularly, PRS-343 induces IL-2 production in the presence of HER2-positive NCI-N87 cells with a potency of about 35 pmol/L (EC₅₀). When the experiment was performed with cell lines expressing basal levels of HER2, no PRS-343-dependent IL-2 induction was observed. Additionally, PRS-343 induces 4-1BB clustering and downstream signaling in a Jurkat NF-κB reporter cell line in the presence of HER2-positive cells with a potency of approximately 50 pmol/L (EC₅₀).

A bell-shaped response was observed for both in the primary T cell activation assay and the Jurkat NF-κB reporter assay, suggesting the response requires the formation of a ternary complex of the tumor cell target HER2, the drug PRS-343, and the T cell receptor 4-1BB and can be disrupted when HER2 and 4-1BB are individually saturated with PRS-343.

### Example 3: Dose escalation study of PRS-343 in patients with HER2+ advanced or metastatic solid tumors

Example 3 provides information on this study for Cohorts 1-11, with additional information for Cohorts 1-13 provided in Example 4.

### A. Study Objectives and Overview

This example describes a Phase 1, open-Label, dose escalation study of PRS-343 in patients with HER2+ advanced or metastatic solid tumors for which standard treatment options are not available, are no longer effective, are not tolerated, or the patient has refused standard therapy. The primary objective of the study is to characterize the safety profile and identify the maximum tolerated dose (MTD) or recommended Phase 2 dose (RP2D) of PRS-343. The secondary objective of the study is to characterize the pharmacokinetic (PK) profile of PRS-343, investigate dosing schedule(s) of PRS-343, obtain preliminary estimates of efficacy of PRS-343, assess the potential immunogenicity of PRS-343, assess the pharmacodynamic (PD) effects of PRS-343, and assess possible PK/safety, PK/PD and PK/efficacy correlations.

PRS-343 was supplied as an aqueous solution in 20 mL glass vials containing 16 mL of PRS-343 drug product at a target protein concentration of 25 mg/mL in 20 mM Histidine, 250 mM Sorbitol, pH 6.3, 0.01% PS80. Enrolled subjects received PRS-343 administered by intravenous (IV) infusion over 2 hours, every 3 weeks (Q3W, 21-day cycles) (Schedule 1) initially. If safety, PK, and PD data suggested a different dosing schedule should be evaluated, Schedule 2 or 3 (dosing every 2 weeks (Q2W) or every 4 weeks (Q4W) in a 28-day cycle, respectively) might be conducted. Separate MTDs might be determined for each schedule evaluated. Patients were allocated to different dose levels in dedicated cohorts (Table 1) and received PRS-343 on Day 1 of each 21-day cycle per Schedule 1, on Days 1 and 15 of each 28-day cycle per Schedule 2, or on Day 1 of each 28-day cycle per Schedule 3.

TABLE 1
PRS-343 dose levels

| Cohort | Dose (mg/kg) |
|---|---|
| 1 | 0.0005 |
| 2 | 0.0015 |
| 3 | 0.005 |
| 4 | 0.015 |
| 5 | 0.05 |
| 6 | 0.15 |
| 7 | 0.5 |
| 8 | 1.0 |
| 9 | 2.5 |
| 10 | 5.0 |
| 11 | 8.0 |

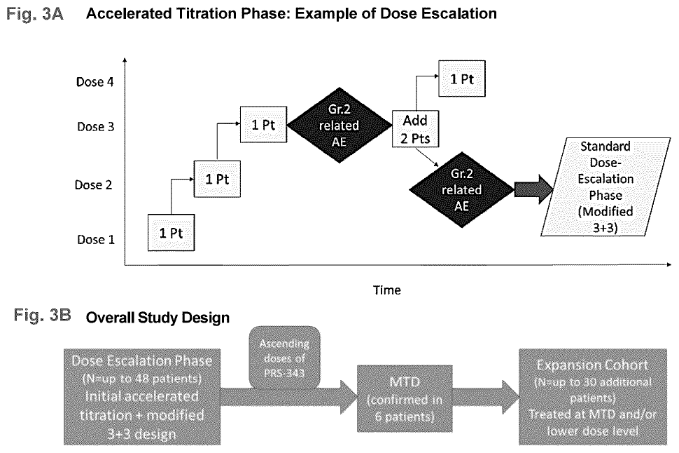

An accelerated titration design was utilized for the initial cohorts (FIG. 3A). Only 1 patient per cohort was enrolled in each escalating dose cohort until a patient experiences a Grade 2 treatment related adverse effect (AE) in Cycle 1, at which time 2 additional patients were enrolled. If a second patient experienced a Grade 2 treatment-related AE, the standard dose-escalation phase was initiated. If neither patient experienced a Grade 2 treatment-related AE, the accelerated titration continued. If a single patient experienced a dose-limiting toxicity (DLT), the modified 3+3 design was initiated (FIG. 3B). In the standard dose-escalation phase, a modified 3+3 design was utilized, allowing 3 or 4 patients to be enrolled in a cohort with expansion up to a total of 6 evaluable patients if a DLT is observed. The modified 3+3 design was scheduled to be initiated for dose levels 8 through 11 and higher (1 mg/kg to 8 mg/kg or higher respectively) if not initiated previously. After each cohort has been enrolled and all patients in the cohort have completed Cycle 1, safety data from all cohorts were reviewed to determine whether to proceed with further dose escalation.

Following identification of a non-tolerated dose, enrollment at the preceding dose would resume until that dose has been administered to 6 evaluable patients. An MTD is defined as the dose level below the dose inducing DLT in 33% of patients. At least 6 evaluable patients must be evaluated in the dose level for it to be called the MTD. Upon establishing MTD, up to 30 additional patients are enrolled in individual expansion cohorts at the MTD and/or at a lower dose level if safety/PD/PK/efficacy data support further evaluation of a lower dose level in order to determine the RP2D.

Subjects were enrolled in the study based on the following criteria: 1. Signed written informed consent obtained prior to performing any study procedure, including screening procedures; 2. Men and women 18 years; 3. Dose escalation: histologically or cytologically confirmed diagnosis of unresectable/locally advanced and/or metastatic HER2+ solid tumor malignancy and for which the standard therapies are not available, are no longer effective, are not tolerated, or have been declined by the patient. Expansion cohort: unresectable/locally advanced or metastatic HER2+ solid tumors considered likely to respond to a HER2-targeted 4-1BB agonist (e.g. gastric/gastroesophageal/esophageal, breast, bladder); 4. Dose escalation and expansion cohort: HER2+ solid tumors documented by clinical pathology report; 5. Patients with breast cancer and gastric and gastroesophageal junction cancer must have received at least 1 prior HER2 targeted therapy for advanced/metastatic disease; 6. Eastern Cooperative Oncology Group (ECOG) performance status (PS) 0-1; 7. Estimated life expectancy of at least 3 months; 8. Dose Escalation: evaluable or measurable disease according to RECIST v1.1. Expansion Cohort (additional 30 patients): measurable disease according to RECIST; 9. Adequate organ function as defined below: a) serum AST and ALT≤3×ULN; if liver meets present ≤5×ULN. b) total serum bilirubin ≤1.5×ULN. C) serum creatinine ≤1.5×ULN OR calculated glomerular filtration rate (GFR) by Cockcroft-Gault formula 50 mL/min. d) Hemoglobin 9 g/dL. e) ANC >1500/mm³. f) Platelet count 75,000/mm³. g) Left ventricular ejection fraction (LVEF) determined by echocardiogram or multigated acquisition scan 50%; 10. Any prior cumulative doxorubicin dose must be ≤360 mg/m²; prior cumulative epirubicin dose must be ≤720 mg/m²; 11. Women of childbearing potential must have a negative serum or urine pregnancy test within 96 hours prior to start of study drug; 12. Women must not be breastfeeding; 13. Women of childbearing potential must agree to follow instruction for method(s) of contraception for the duration of treatment with study drug PRS-343 plus 90 days post-treatment completion; 14. Males who are sexually active with women of childbearing potential must agree to follow instructions for method(s) of contraception for the duration of treatment with study drug PRS-343 plus 90 days post-treatment completion.

Additionally, subjects who met any of the following criteria were not enrolled: 1. Known uncontrolled central nervous system (CNS) metastases and/or carcinomatous meningitis. Note: Patients with previously treated brain metastases may participate provided they are stable (without evidence of progression by imaging for at least 4 weeks prior to the first dose of study treatment and any neurologic symptoms have returned to baseline), have no evidence of new or enlarging brain metastases, and are clinically stable off steroids for at least 7 days prior to study treatment. Carcinomatous meningitis precludes a patient from study participation regardless of clinical stability; 2. History of acute coronary syndromes, including myocardial infarction, coronary artery bypass graft, unstable angina, coronary angioplasty or stenting within past 24 weeks; 3. History of or current Class II, III or IV heart failure as defined by the New York Heart Association (NYHA) functional classification system; 4. History of ejection fraction drop below the lower limit of normal with trastuzumab and/or pertuzumab; 5. Medical, psychiatric, cognitive or other conditions that compromise the patient's ability to understand the patient information, to give informed consent, to comply with the study protocol or to complete the study; 6. Any severe concurrent disease or condition (includes active infections, cardiac arrhythmia, interstitial lung disease) that in the judgment of the investigator would make study participation inappropriate for the patient; 7. Previously known active infection with human immunodeficiency virus (HIV); or hepatitis B or hepatitis C infection. Patients with positive hepatitis B core antibody (HBcAb) require assessment and monitoring of virus deoxyribonucleic acid (DNA) status; patients with positive hepatitis C virus (HCV) core antibody can enroll if HCV ribonucleic acid (RNA) is negative; 8. History of infusion reactions to any component/excipient of PRS-343; 9. Systemic steroid therapy (>10 mg daily prednisone or equivalent) or any other form of immunosuppressive therapy within 7 days prior to the first dose of study treatment (Note: topical, inhaled, nasal and ophthalmic steroids are not prohibited); 10. Autoimmune disease that has required systemic treatment in the past (i.e., with use of disease-modifying agents, corticosteroids, or immunosuppressive drugs). Replacement therapy (e.g., thyroxine, insulin, or physiologic corticosteroid replacement therapy for adrenal or pituitary insufficiency, etc.) is allowed; 11. Has not recovered from the adverse effect of previous anticancer treatments to pretreatment baseline or Grade 1 except for alopecia, anemia (hemoglobin levels must meet the study inclusion criteria) and peripheral neuropathy (which must have recovered to s Grade 2) nausea and diarrhea if anti-emetic and anti-diarrheal treatment hasn't been exhausted; 12. History of a second primary cancer with the exception of 1) curatively treated nonmelanomatous skin cancer, 2) curatively treated cervical or breast carcinoma in situ, or 3) other malignancy with no known active disease present and no treatment administered during the last 2 years; 13. Receipt of investigational treatment within 3 weeks of scheduled Cycle 1 Day 1 (C1D1) dosing; 14. Receipt of cytotoxic chemotherapy within 3 weeks (6 weeks for nitrosoureas and mitomycin C) of scheduled C1D1 dosing; 15. Receipt of radiation therapy within 3 weeks of scheduled C1D1 dosing, unless the radiation comprised a limited field to non-visceral structures (e.g., limb bone metastasis); 16. Receipt of treatment with immunotherapy, biological therapies, targeted small molecules, hormonal therapies within 3 weeks of scheduled C1D1 dosing; 17. Receipt of trastuzumab or ado-trastuzumab emtansine or any other experimental drug that engages the same epitope as trastuzumab within 4 weeks of scheduled C1D1 dosing; 18. Concurrent enrollment in another therapeutic clinical trial; 19. Major surgery within 3 weeks of scheduled C1D1 dosing.

### B. Study Procedures

Subjects with unknown HER2 status were consented separately in a pre-screening visit in order to undergo HER2 testing prior to screening. All subjects were screened within 28 days priorto administration of the drug (Day −28 to −1) to confirm that they meet the study selection criteria and evaluated for baseline (Day 1 predose).

In Schedule 1, subjects received the first dose of PRS-343 on Day 1 of Cycle 1 followed by subsequent doses on Day 1 of each cycle (every 3 weeks). Patient assessments occurred on Days 1, 2, 3, 4, 8, and 15 of Cycle 1; Days 1 and 2 through 8 of Cycle 2; Days 1, 2, 3, 4, 8, and 15 of Cycle 3; then on Day 1 of all subsequent cycles. Assessments also occurred on Day 21 (±7 days) of Cycles 2, 4, 6, and 8 and Day 21 of every 4 cycles (12 weeks [±7 days]) thereafter.

In Schedule 2, subjects received the first dose of PRS-343 on Day 1 of Cycle 1 followed by a dose on Day 15 of Cycle 1 and subsequent doses on Days 1 and 15 of each cycle (every 4 weeks). Patient assessments occurred on Days 1, 2, 3, 4, 8, 15, and 22 of Cycle 1; Days 1, 2 through 8, and 15 of Cycle 2; Days 1, 2, 3, 4, 8, and 15 of Cycle 3; then on Days 1 and 15 of all subsequent cycles. Assessments also occurred on Day 28 (±7 days) of Cycles 2, 4, and 6 and Day 28 of every 3 cycles (12 weeks [±7 days]) thereafter.

In Schedule 3, subjects received the first dose of PRS-343 on Day 1 of Cycle 1 followed by subsequent doses on Day 1 of each cycle (every 4 weeks). Patient assessments occurred on Days 1, 2, 3, 4, 8, and 15 of Cycle 1; Days 1 and 2 through 8 of Cycle 2; Day 1 of Cycle 3; Days 1, 2, 3, 4, 8, and 15 of Cycle 4; then on Day 1 of all subsequent cycles. Assessments also occurred on Day 28 (±7 days) of Cycles 2, 4, and 6 and Day 28 of every 3 cycles (12 weeks [±7 days]) thereafter.

Dose-limiting toxicities (DLTs) were reported during the first cycle of each schedule (e.g., 21 days after the first dose in Cycle 1 for Schedule 1). Subjects were monitored for safety throughout the study. Dosing would continue until criteria for study drug discontinuation were met (disease progression or withdrawal from the study). The subjects would return for safety follow-up on Day 30 (±3 days) after they received the last dose.

### C. Endpoints and Assessments

The primary endpoint of this study is incidence and severity of adverse effects (AEs) graded according to the National Cancer Institute Common Terminology Criteria for Adverse Events (NCI CTCAE) version 4.03. The safety and tolerability of PRS-343 was also assessed based on vital signs, physical examinations, ECOG performance status, electrocardiogram (ECG), and laboratory safety tests on an ongoing basis during the study.

Patients were monitored for AEs during study participation (beginning at the time study drug is first administered) and until 30 days after the last dose of study drug. Any ongoing serious adverse events (SAEs) were followed until resolution or stabilization. Assessments of vital signs included body temperature, systolic and diastolic blood pressure readings (mm Hg), pulse (beats per minute [BPM]), and respiratory rate (breaths rate per minute [BRPM]). Triplicate 12-lead ECG measurements were performed at pre-determined time points and collected within 10 minutes of the scheduled collection time, prior to the blood collection if collected at the same time. The mean of the triplicate ECG measurements performed pre-dose on Day 1 served as the patient's baseline corrected QT (QTc) value for all post-dose comparisons. Blood and urine samples were collected for laboratory assessments, including hematology, coagulation, serum chemistry, urinalysis, pregnancy screen, left-ventricular ejection fraction, cytokines, and washout blood sample.

For the primary endpoint, all subjects who received at least 1 dose of PRS-343 were included in the safety analyses. Safety data are presented in tabular and/or graphical format and summarized descriptively by dose cohort and time as appropriate. Absolute value data and changes from baseline data are summarized as appropriate.

The secondary endpoints of this study are serum PK parameters; PK and safety profile for Schedule 1, as well as Schedule 2 and Schedule 3, if applicable; tumor responses; duration of response; disease control rate; presence and/or concentration of anti-PRS-343 antibodies (ADAs); and PD markers.

Venous blood samples for the PK analysis and ADA assessment were collected at pre-determined time points. PK profiles to assess PK properties of single agent PRS-343 were collected from all enrolled subjects. The PK parameters determined for PRS-343 include, but are not limited to, the area under the curve (AUC), AUC₂₄h, AUCi_{nf}, Cₘₐₓ, time to maximum dose concentration (tₘₐₓ), and terminal half-life (t₁/2) of PRS-343. Tumor assessments, including tumor markers, will be performed at pre-determined time points, and tumor response and progression were assessed according to RECIST, Version 1.1. PD marker were assessed by quantifying lymphocyte subtypes or markers in tumor biopsies or peripheral blood and cytokine levels in plasma at pre-determined time points, prior, during, and after the duration of the dosing. The PD markers measured as available and feasible include, but are not limited to, IHC cell subsets (e.g., CD8, CD4, PDL-1, Ki67) assessed in pre-treatment (prior to Cycle 1, Day 1 dosing) and on-treatment tumor biopsies (Cycle 2, within Days 2-8), 4-1BB, soluble HER2, and IFN-γ assessed in pre-treatment (prior to Cycle 1, Day 1 dosing) and on-treatment plasma samples, CD8 T cells, CD4 T cells assessed in pre-treatment (prior to Cycle 1, Day 1 dosing) and on-treatment blood samples, and IHC cell subsets (e.g., CD8, CD4, PDL-1, Ki67) assessed in post-relapse (optional) tumor biopsies. Additionally, the PK/PD relationship and relationship to tumor response are explored.

### Example 4. Dose escalation study of PRS-343 in patients with HER2+ advanced or metastatic solid tumors

This example provides information on this study for Cohorts 1-13 and interim data for Cohorts 1-11.

### A. Study Objectives and Overview

The study objectives were as described in Example 3.

Patients were allocated to different dose levels in dedicated cohorts including additional Cohorts 12 and 13 (Table 2) and received PRS-343 administered by intravenous (IV) infusion over 2 hours, every 3 weeks (Q3W) (Schedule 1) initially. If safety, PK, and PD data suggested a different dosing schedule should be evaluated, Schedule 2 (every 2 weeks, Q2W) or Schedule 3 (every week, Q1W) might be conducted. Separate MTDs may be determined for each schedule evaluated. Separate MTDs might be determined for each schedule evaluated.

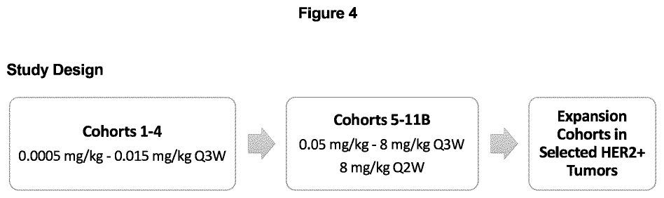

A 1+3 dose escalation design was utilized for Cohorts 1 through 4 (0.0005 mg/kg to 0.015 mg/kg, respectively), and a 3+3 design was used for Cohorts 5 through 11 (0.05 mg/kg to 8 mg/kg, respectively). At the Cohort 11 (8 mg/kg) and above until Cohort 13 (18 mg/kg), the three dose schedules—Q1W, Q2W, and Q3W—were studied (FIG. 4).

TABLE 2
PRS-343 dose levels

| Cohort | Dose (mg/kg) |
|---|---|
| 1 | 0.0005 |
| 2 | 0.0015 |
| 3 | 0.005 |
| 4 | 0.015 |
| 5 | 0.05 |
| 6 | 0.15 |
| 7 | 0.5 |
| 8 | 1 |
| 9 | 2.5 |
| 10 | 5.0 |
| 11 | 8 |
| 12 | 12 |
| 13 | 18 |

### B. Study Procedures

The study procedures were as described in Example 3, except for that in Schedule 3, subjects received the first dose of PRS-343 on Day 1 of Cycle 1 followed by doses on Days 8 and 15 of Cycle 1 and subsequent doses on Days 1, 8, and 15 of each cycle (every 3 weeks). Patient assessments for Schedule 3 occurred on Days 1, 2, 3, 4, 8, and 15 of Cycle 1; Days 1, 2, 3, 4, 8, and 15 and Day 21 (±7 days) of Cycle 2; then on Days 1, 8, and 15 of all subsequent cycles. Assessments also occurred on Day 21 (±7 days) of Cycles 4, 6, 8, and every 3 cycles thereafter.

Particularly, patients were assessed for tumor response/progression per RECIST v1.1. For Schedule 1, patients are assessed every 6 weeks for the initial 24 weeks of dosing (first 8 cycles). After the week 24 scans, tumor assessments are conducted every 12 weeks. For Schedules 2 and 3, patients are assessed every 8 weeks for the initial 24 weeks of dosing (first 6 cycles for Schedule 2 and first 8 cycles for Schedule 3). After the week 24 scans, tumor assessments are conducted every 12 weeks.

### C. Endpoints and Assessments

The study procedures were as described in Example 3.

### D. Data Analysis/Methods

(i) PK

Preliminary pharmacokinetic (PK) results of PRS-343 are available at dose levels of 0.0005, 0.0015, 0.005, 0.015, 0.05, 1, 2.5, 5 and 8 mg/kg administered every 3 weeks (Q3W) and 8 mg/kg every 2 weeks (Q2W). PRS-343 was administered as a 2-hour intravenous infusion. In the Q3W dosing regimen, PRS-343 single dose and multiple dose pharmacokinetics were characterized after the first dose (Cycle 1 Day 1) and third dose (Cycle 3 Day 1), respectively. In the Q2W dosing regimen, PRS-343 single dose and multiple dose pharmacokinetics were characterized after the first dose (Cycle 1 Day 1) and fifth dose (Cycle 3 Day 1), respectively. Serum concentration data and planned times were analyzed using non-compartmental methods and preliminary PK results are presented here.

(ii) Anti-Drug Antibody Formation

Given the relatively small sample size per cohort and more data is being collected from ongoing studies, anti-drug antibody results and conclusions should be interpreted as preliminary. Immunogenicity samples collected were analyzed using a validated assay for anti-PRS-343 antibodies (ADA) and, if the sample was confirmed positive for ADA, titer value was determined. The lowest measurable titer value of the assay was 50. A patient was considered to be ADA negative, if no ADA were detected in any immunogenicity sample. If ADA were detected, depending on the maximum titer value observed, the patient was either categorized as low-titer (value below limit of quantification, values of 50 and 150) or high-titer (any value greater than 150). Titer value cutoff of 150 was used to categorize ADA positive patients, in part, based on significant impact of titer values greater than 150 on PRS-343 pharmacokinetics.

(iii) Efficacy

Efficacy was evaluated by tumor response for patients with measurable or evaluable disease as assessed by the Investigators using RECIST version 1.1 (Appendix 1). Duration of response was calculated for patients who achieve a complete response (CR) or partial response (PR) and was defined as the time from the date of first documented response (CR or PR) to the date of documented progression or death after achieving response. Disease control rate was defined as the percentage of patients who have achieved CR, PR, or SD (stable disease) lasting at least 12 weeks.

(iv) PD—Quantification of treatment induced changes of CD8 T cells numbers

In order to investigate whether PRS-343 is an active drug, treatment induced PD marker changes were assessed by quantifying CD8+ T cells in tumor biopsies in pre-treatment (prior to Cycle 1, Day 1 dosing) and on-treatment tumor biopsies (Cycle 2, within Days 2-8) by immunohistochemistry (IHC) staining.

Core needle biopsies were taken as specified by the clinical protocol, formaldehyde fixed and paraffin embedded, and sectioned in 3 uM sections for chromogenic IHC with anti-CD8 antibodies as well as other markers. Pathology guided digital annotations of tumor cells and stroma areas were performed. CD8+ T cells were counted per mm² of tumor cells, tumor stroma, and full tumor tissue (tumor stroma+tumor cells).

### E. Preliminary Results

A total of 52 patients have been treated with PRS-343 administered as a single agent (Tables 3 and 4). The median age at treatment is 61 years and 32 (62%) of the patients were female. Forty (77%) of the treated patients had ECOG PS of 1 and the rest had a PS of 0. This was a previously heavily treated population of patients with 20 or 38% having received 5+ lines of therapy, 10 (19%) having received 4 lines of therapy and 11 or 21% having received 3 lines of therapy. Of the wide range of tumor types studied 19 (37%) had gastroesophageal cancer, 13 (25%) had breast cancer and 6 (12%) had gynecological cancer.

TABLE 3
Current enrolment of PRS-343 study

| Cohort | Dose & regimen | No. Patients |
|---|---|---|
| 1 | 0.0005 mg/kg Q3W | 1 |
| 2 | 0.0015 mg/kg Q3W | 1 |
| 3 | 0.005 mg/kg Q3W | 1 |
| 4 | 0.015 mg/kg Q3W | 2 |
| 5 | 0.05 mg/kg Q3W | 2 |
| 6 | 0.15 mg/kg Q3W | 5 |
| 7 | 0.5 mg/kg Q3W | 7 |
| 8 | 1 mg/kg Q3W | 6 |
| 9 | 2.5 mg/kg Q3W | 6 |
| 10 | 5 mg/kg Q3W | 9 |
| 11 | 8 mg/kg Q3W | 6 |
| 11B | 8 mg/kg Q2W | 6 |
| TBD (data driven) | 8 mg/kg Q2W | |
| Total | | 52 |

TABLE 4
Baseline characteristics of enrolled subjects

| | n (%) | |
|---|---|---|
| Characteristic | | |
| Age, Median (range) | 61 | (29-92) |
| Gender | | |
| F | 32 | (62%) |
| M | 20 | (38%) |
| ECOG PS | | |
| 0 | 12 | (23%) |
| 1 | 40 | (77%) |
| Prior Therapy Lines | | |
| 1 | 6 | (12%) |
| 2 | 5 | (10%) |
| 3 | 11 | (21%) |
| 4 | 10 | (19%) |
| 5+ | 20 | (38%) |
| Average HER2 Targeting Treatments | | |
| Breast | | |
| Gastric | | |
| Primary Cancer Type | | |
| Biliary | 2 | (4%) |
| Bladder | 2 | (4%) |
| Breast | 13 | (25%) |
| Colorectal | 5 | (10%) |
| Gall Bladder | 2 | (4%) |
| Gastroesophageal | 19 | (37%) |
| Gynecological | 6 | (12%) |
| Pancreatic | 1 | (2%) |
| Other - Salivary Duct | 1 | (2%) |
| Other - Melanoma | 1 | (2%) |

Of the treatment related adverse events reported, the most common were infusion related reactions (10 incidents or 9% of all TRAE), fatigue (10 incidents or 9% of all TRAEs) and chills in 7 or 6% of all reported TRAEs (Table 5).

TABLE 5
Treatment-related adverse

| Occurred in ≥1 Patient | N = 111 \| n (%) | % Grade 3 |
|---|---|---|
| Infusion Related Reaction | 10 (9%) | 2 (2%) |
| Fatigue | 10 (9%) | 1 (1%) |
| Chills | 7 (6%) | 0 |
| Flushing | 7 (6%) | 3 (3%) |
| Nausea | 7 (6%) | 0 |
| Diarrhea | 7 (6%) | 0 |
| Vomiting | 6 (5%) | 0 |
| Non-Cardiac Chest Pain | 5 (6%) | 1 (1%) |

(i) Preliminary PK Results

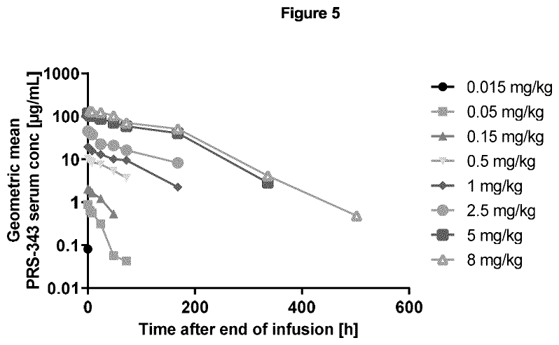

Single dose geometric mean serum concentrations are shown in FIG. 5 and preliminary PK parameters are shown in Table 6.

TABLE 6
Preliminary geometric mean (% CV) single dose
(Cycle 1) PRS-343 pharmacokinetic parameters

| | Dose & | Number of | Cₘₐₓ | tₘₐₓ | AUC₂₄ | AUC_{INF} | t_{1/2} |
|---|---|---|---|---|---|---|---|
| Cohort | regimen | Patients | (μg/mL) | (h) ¹ | (μg × h/mL) | (μg × h/mL) | (h) |
| 1 | 0.0005 | N = 1 | BLQ | | | | |
| | mg/kg Q3W | | | | | | |
| 2 | 0.0015 | N = 1 | BLQ | | | | |
| | mg/kg Q3W | | | | | | |
| 3 | 0.005 | N = 1 | BLQ | | | | |
| | mg/kg Q3W | | | | | | |
| 4 | 0.015 | N = 2 | 0.08 | 0.08 | not | not | not |
| | mg/kg Q3W | | (56%) | (0.08-0.08) | available | available | available |
| 5 | 0.05 | N = 2 | 0.89 | 0.08 | 13 | 21 | 14.8 |
| | mg/kg Q3W | | (53%) | (0.08-0.08) | (21%) | (2%) | (32%) |
| 6 | 0.15 | N = 5 | 2.09 | 0.5 | 39 | 78 | 23.5 |
| | mg/kg Q3W | | (54%) | (0.08-8) | (53%) | (68%) | (19%) |
| 7 | 0.5 | N = 6 | 10.35 | 0.2 | 214 | 793 | 52.9 |
| | mg/kg Q3W | | (23%) | (0.08-8) | (28%) | (65%) | (35%) |
| 8 | 1 | N = 6 | 19.46 | 0.08 | 376 | 1657 | 64.3 |
| | mg/kg Q3W | | (28%) | (0.08-0.08) | (27%) ² | (41%) ² | (30%) ² |
| 9 | 2.5 | N = 6 | 45.34 | 0.08 | 928 | 4530 | 74.8 |
| | mg/kg Q3W | | (35%) | (0.08-0.08) | (34%) | (77%) | (50%) |
| 10 | 5 | N = 7 | 119.74 | 0.1 | 2480 | 17033 | 118 |
| | mg/kg Q3W | | (15%) | (0.08-4) | (17%) | (53%) | (45%) |
| 11 | 8 | N = 5 | 146.39 | 0.4 | 3243 | 23930 | 104 |
| | mg/kg Q3W | | (25%) | (0.08-4) | (19%) | (39%) | (51%) |
| 11B | 8 | N = 6 | 142.59 | 0.2 | 3137 | 21763 | 106 |
| | mg/kg Q2W | | (31%) | (0.08-8) | (33%) | (35%) | (31%) |

BLQ below limit of quantification
¹ median (range)
² n = 5

Serum PRS-343 concentration were very low or below the limit of quantitation at the 0.0005 mg/kg to 0.05 mg/kg dose levels. At the 0.15 mg/kg dose level, serum PRS-343 concentrations were measurable for 3 days postdose and at the 0.5 and 1 mg/kg dose level, serum PRS-343 concentrations were measurable up to 14 days postdose in several patients. Starting at 2.5 mg/kg dose level, serum concentrations were measurable throughout the 3-week dosing interval in several patients.

Maximum serum concentration of PRS-343 were typically observed within 5 minutes after end of infusion. In few patients, maximum serum concentrations were observed at 4 or 8 hours after the end of infusion; however, these concentrations were not substantially greater than end of infusion concentrations, except from one patient where end of infusion concentration was below limit of quantification.

In the dose range 0.5 mg/kg to 8 mg/kg, PRS-343 Cₘₐₓ and AUC₂₄ increased at a dose proportional manner. PRS-343 exhibited dose proportional AUC_{INF} at the 2.5 mg/kg to 8 mg/kg dose levels. Variability in PRS-343 pharmacokinetic parameters was low to moderate. At the 2.5 mg/kg and higher dose levels where sufficient data points were available for reliable estimation of half-life, average half-life of at least 3 days was estimated. At the highest dose of 8 mg/kg Q3W, average PRS-343 half-life was estimated to be 104 hours (4.3 days).

Cycle 3 multiple dose pharmacokinetic results are available in a limited number of patients and are discussed in the context of immunogenicity results (ADA formation).

(ii) Preliminary ADA Formation Results

Incidence of ADA in patients with at least one postdose sample analyzed for ADA is summarized in Table 7.

TABLE 7
Incidence of anti-PRS-343 antibodies (anti-drug antibodies, ADA)

| | | Number of | | Number (%) | Number (%) | |
|---|---|---|---|---|---|---|
| | | patients with | Number (%) | of patients | of patients | Highest |
| | | at sample | of patients | with low- | with high- | titer |
| | | least 1 | with no | titer ADA | titer ADA | reported in |
| | Dose & | postdose | measurable | (titer ≤ | (titer > | each dose |
| Cohort | regimen | sample | ADA | 150) | 150) | level |
| 1 | 0.0005 | 1 | 0 (0%) | 0 (0%) | 1 (100%) | 984,000 |
| | mg/kg Q3W | | | | | |
| 2 | 0.0015 | 1 | 1 (100%) | 0 (0%) | 0 (0%) | n/a |
| | mg/kg Q3W | | | | | |
| 3 | 0.005 | 1 | 1 (100%) | 0 (0%) | 0 (0%) | n/a |
| | mg/kg Q3W | | | | | |
| 4 | 0.015 | 2 | 2 (100%) | 0 (0%) | 0 (0%) | n/a |
| | mg/kg Q3W | | | | | |
| 5 | 0.05 | 1 | 0 (0%) | 0 (0%) | 1 (100%) | 12,100 |
| | mg/kg Q3W | | | | | |
| 6 | 0.15 | 5 | 4 (80%) | 0 (0%) | 1 (20%) | 1,350 |
| | mg/kg Q3W | | | | | |
| 7 | 0.5 | 6 | 1 (16.7%) | 1 (16.7%) | 4 (66.7%) | 8,860,000 |
| | mg/kg Q3W | | | | | |
| 8 | 1 mg/kg | 5 | 2 (40%) | 0 (0%) | 3 (60%) | 36,500 |
| | Q3W | | | | | |
| 9 | 2.5 mg/kg | 5 | 0 (0%) | 3 (60%) | 2 (40%) | 109,000 |
| | Q3W | | | | | |
| 10 | 5 mg/kg | 5 | 3 (60%) | 1 (20%) | 1 (20%) | 450 |
| | Q3W | | | | | |
| 11 | 8 mg/kg | 5 | 2 (40%) | 2 (40%) | 1 (20%) | 36,500 |
| | Q3W | | | | | |
| 11B | 8 mg/kg | 3 | 1 (33.3%) | 1 (33.3%) | 1 (33.3) | 4,050 |
| | Q2W | | | | | |

| | | | | |
|---|---|---|---|---|
| Cohorts 9, 10, 11 & 11B | 18 | 6 (33.3%) | 7 (38.9%) | 5 (27.8%) |
| All cohorts | 40 | 17 (42.5%) | 8 (20%) | 15 (37.5%) |

Out of 40 patients treated with PRS-343 at doses ranging from 0.0005 mg/kg to 8 mg/kg with at least one postdose immunogenicity sample, 17 patients were ADA negative. ADA was detected in at least one post-dose sample in the remaining 23 patients with 8 patients considered to have low titers and 15 patients considered to have high titers.

Based on overall safety profile, further evaluation of PRS-343 is expected to continue at higher dose levels. Therefore, immunogenicity data are also summarized for the three highest dose levels (currently considered to be clinically relevant) of 2.5, 5 and 8 mg/kg. In Cohorts 9 and above, out of 18 patients, 6 patients were ADA negative, 7 patients were ADA positive with low-titer and 5 patients were ADA positive with high-titer.

In most of ADA positive patients, ADA was detected as early as 14 days after the first dose, the first time point of immunogenicity assessment.

Effect of ADA on pharmacokinetics of PRS-343 exposures in 11 patients with preliminary pharmacokinetic data in both Cycles 1 and 3 along with ADA titers, if applicable, are shown in Table 8.

TABLE 8
Exposure of PRS-343 in patients with both Cycle 1 and Cycle 3 preliminary PK data

| | | Cycle 1 | Cycle 3 | |
|---|---|---|---|---|
| | PRS-343 dose | PRS-343 | PRS-343 | |
| | and dosing | AUC(0-t), | AUC(0-t), | ADA result and titer, if |
| Subject ID | regimen | ug × h/mL | ug × h/mL | applicable, up to Cycle 4 Day 1 |
| 111-001 | 0.15 mg/kg Q3W | 114 | 90.6 | C1D15: ADA negative |
| | | | | C3D1: ADA negative |
| | | | | C4D1: ADA negative |
| 111-002 | 0.15 mg/kg Q3W | 99.3 | 0.847 | C1D15: Titer 50 |
| | | | | C3D1: Titer 1350 |
| | | | | C4D1: Titer 1350 |
| 104-005 | 0.5 mg/kg Q3W | 1790 | 61.7 | C1D15: ADA negative |
| | | | | C3D1: Titer 1350 |
| | | | | C4D1: Titer 12100 |
| 106-001 | 1 mg/kg Q3W | 1320 | 200 | C1D15: Titer 12100 |
| | | | | C3D1: Titer 450 |
| | | | | C4D1: not available |
| 107-004 | 1 mg/kg Q3W | 171 | 0.592 | C1D15: not available |
| | | | | C3D1: not available |
| | | | | C3 unscheduled: Titer 4050 |
| | | | | C4D1: Titer 150 |
| | | | | C5D1: Titer 36500 |
| 108-002 | 2.5 mg/kg Q3W | 7243 | 1857 | C1D15: ADA negative |
| | | | | C3D1: ADA negative |
| | | | | C4D1: not available |
| 103-013 | 5 mg/kg Q3W | 12537 | 1968 | C1D15: Titer <50 |
| 103-015 | 5 mg/kg Q3W | 13578 | 1516 | C3D1: not available |
| | | | | C4D1: not available |
| | | | | No data available |
| 103-009 | 8 mg/kg Q3W | 35192 | 49990 | C1D15: ADA negative |
| | | | | C3D1: ADA negative |
| | | | | C4D1: ADA negative |
| 108-005 | 8 mg/kg Q3W | 25132 | 4362 | C1D15: ADA negative |
| | | | | C3D1: ADA negative |
| | | | | C4D1: Titer 1350 |
| 103-012 | 8 mg/kg Q2W | 26353 | 217 | No data available |
| 104-006 | 8 mg/kg Q2W | 28332 | 8392 | C1D15: Titer 50 |
| | | | | C2D15: Titer 50 |
| 107-012 | 8 mg/kg Q2W | 15227 | 1946 | Titer 150 C3D1: C4D1: Not available |
| | | | | No data available |
| 110-003 | 8 mg/kg Q2W ¹ | 17219 | 1330 | C1D15: ADA negative |
| | | | | C2D15: not available |
| | | | | C3D1: not available |
| | | | | C4D1: not available |

¹ Cycle 1 Day 1 PRS-343 dose: 481.6 mg; Cycle 3 Day 1 PRS-343 dose: 309 mg

Evaluation of relationship between decrease in Cycle 3 PRS-343 exposure and ADA titer values determined up to Cycle 4 Day 1 indicates that substantially lower PRS-343 exposure in Cycle 3 is associated with titer values of at least 450 with the exception of a single patient (Subject ID 104-006).

In patients without ADA, Cycle 1 and Cycle 3 exposure were comparable indicating no accumulation after Q3W administration. A patient (Subject ID 108-002) had lower exposure in Cycle 3 without ADA detected until Cycle 4 Day 1.

(iii) PK/PD Relationship

Based on the preclinical dataset demonstrating maximum activity of PRS-343 was observed in vitro at 10 nM (=2 pg/mL) and the assumption that 10% of the drug gets to the tumor, a serum concentration of 20 pg/mL was predicted to be needed for full activity of PRS-343 in the tumor.

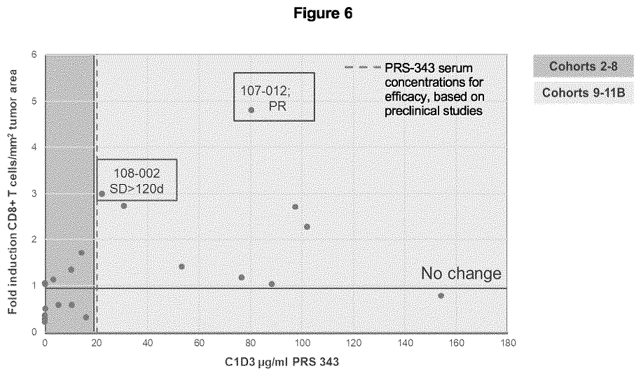

FIG. 6 shows a drug exposure/PD relationship graph. For Cohorts 1 to 8 (dose levels ranging from 0.0005 mg/kg to 1 mg/kg), the drug exposure is below 20 pg/mL. From Cohort 9 onwards (dose levels at 2.5 mg/kg and above), plasma drug levels are above 20pg/ml.

From Cohort 9 onwards (dose levels at 2.5 mg/kg and above), strong increases in CD8+ T cell infiltration were observed in some patients, most notably for those with long lasting stable disease (SD) (108-002) and partial response (PR) (107-012) who showed a 3- and 4.8-fold induction of CD8+ T cells on treatment, respectively (FIG. 6).

These results demonstrate the 4-1BB arm activity of PRS-343 can lead to increased levels of CD8+ T cell in the tumor benefiting patients, and indicate dose levels at 2.5 mg/kg and above are in the active dose range as evidenced by the strong immune-stimulatory effect of PRS-343.

(iv) Drug Activity and Emergent Determinants of Response

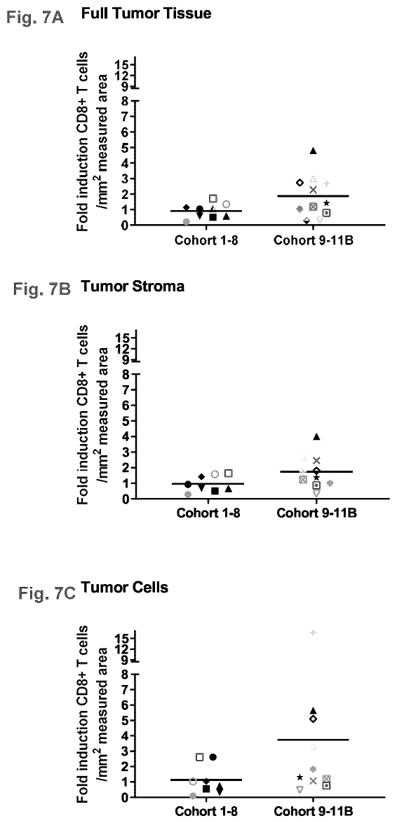

Based on the observation that more pronounced increase of CD8+ T cells is measured in patients receiving doses 2.5 mg/kg from Cohort 9 onwards (FIG. 7), PRS-343 induced increases in CD8+ T cell numbers were quantified for higher dose cohorts (Cohorts 9-11B) and compared to lower dose cohorts (Cohorts 1-8).

On average, in full tumor tissue, a 2-fold induction of CD8+ T cells in high dose cohorts as compared to low dose cohorts were observed (FIG. 7). Additionally, CD8+ T cell changes are more pronounced in the HER2+ tumor cells (FIG. 7B) as compared to the tumor stroma and full tumor tissue (FIGS. 7A and 7C) consistent with the mode of action of a HER2/4-1BB bispecific disclosed herein which forces a proximity of HER2+ tumor cells and 4-1BB expressing CD8+ T cells.

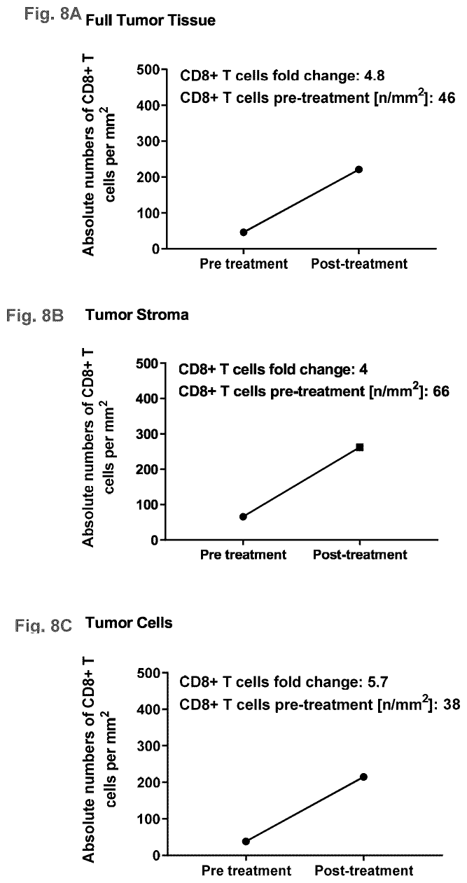

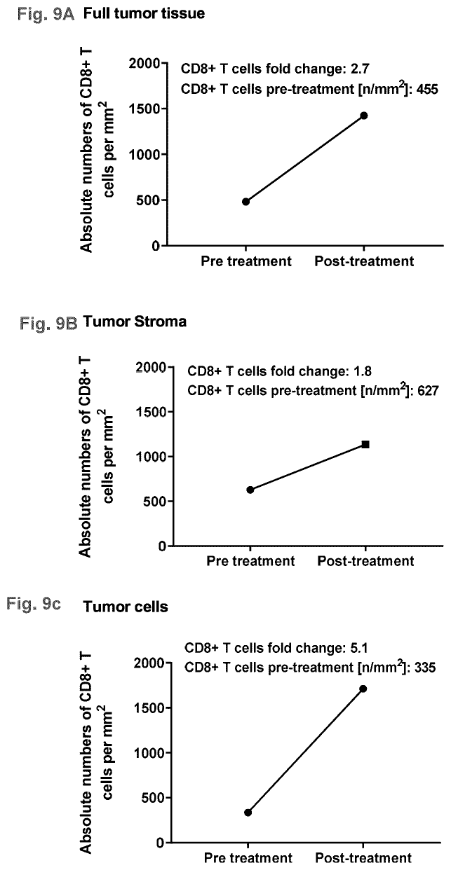

Further evidence for drug activity stems from data showing that the CD8+ T cell increases are particularly strong in patients benefiting from the treatment, e.g., patient 108-002 with SD>120d (FIGS. 7A and 9) and patient 107-012 with PR (FIGS. 7A and 8).

Exemplary results of the responding patients 107-012 and 108-002 are shown in FIG. 8 and FIG. 9, respectively. Surprisingly, patient 107-012 showed very low CD8+ T cell numbers in biopsies prior to treatment—46 CD8+ T cells/mm² of full tumor tissue, which increased on treatment by 4.6-fold. The fold increase of CD8+ T cells for both patients were more pronounced in tumor cells (5.7-fold for patient 107-012 and 5.1-fold for patient 108-002) as compared to tumor stroma (4-fold for patient 107-012 and 1.9-fold for patient 108-002), which is consistent with the mode of action of a HER2/4-1BB bispecific molecule disclosed herein, driving a proximity relationship of HER2+ tumor cells with a 4-1BB+/CD8+ T cells.

Current literature evidence suggests, depending on the indication, that check point molecules need 250 CD8+ T cells/mm2 in tumor tissue prior to treatment for the drugs to show efficacy in patients (Blando et al., 2019, Chen et al., 2016, Tumeh et al., 2014). Surprisingly, responding patients 107-012 and 103-012 in Cohort 11B showed very low numbers of CD8+ T cells in biopsies prior to treatment—46 and 110 CD8+ T cells/mm2 in tumor tissue, respectively. This suggests that a 4-1BB based bispecific drug as disclosed herein, can produce patient benefit where standard check point drugs cannot.

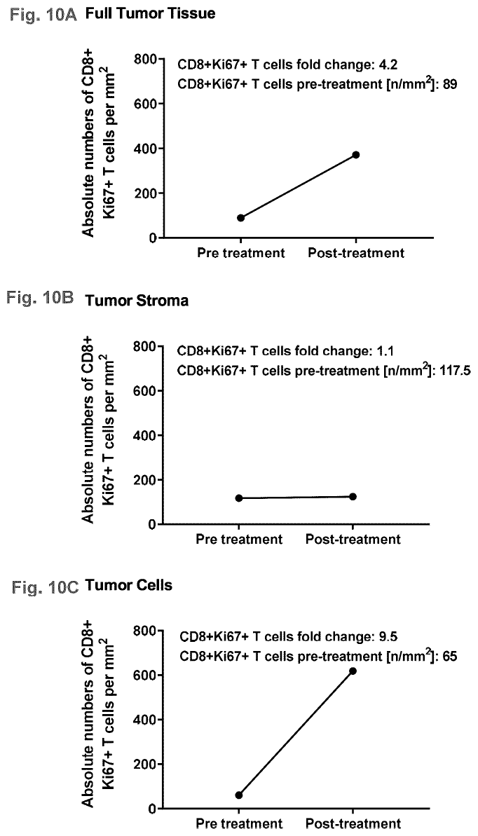

Exemplary results on CD8+Ki67+ T cell expansion of the responding patient 108-002 are also presented herein (FIG. 10). Notably, the CD8+Ki67+ T cell expansion was only observed in tumor cells (FIG. 10C) but not in tumor stroma (FIG. 10B), further suggesting a 4-1BB based bispecific drug as described herein activates CD8+ T cells only in the vicinity of tumor cells.

(v) Tumor Response

From pre-clinical data and PK/PD correlations in the study population, it was estimated that 20 pg/mL is the serum concentration of the drug which results in an efficacious dose in the tumor microenvironment. This serum concentration was reached in Cohort 9. Eighteen evaluable patients are present in Cohorts 9-11B, of which 2 patients recorded a partial response and 8 patients showed stable disease (Table 9).

TABLE 9
Summary of Response at Active Dose Range of PRS-343

| Cohort | 11B | 11 | 10 | 9 | |
|---|---|---|---|---|---|
| Best Response | 8 mg/kg, Q2W | 8 mg/kg, Q3W | 5 mg/kg, Q3W | 2.5 mg/kg, Q3W | Total |
| Response | 5 | 4 | 4 | 5 | 18 |
| Evaluable Patients | | | | | |
| CR/PR | —/2* | —/— | —/— | —/— | —/2 |
| SD | 3 | 2 | 1 | 2 | 8 |
| PD | — | 2 | 3 | 3 | 8 |
| ORR | 40% | 0% | 0% | 0% | 11% |
| DCR | 100% | 50% | 25% | 40% | 55% |

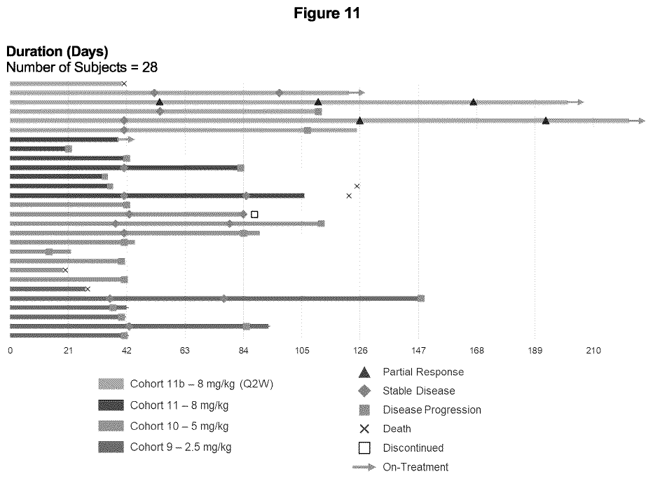

FIG. 11 depicts treatment duration of patients on PRS-343. In Cohort 9 (2.5 mg/kg, Q3W), patients stayed on study (defined as the time between Cycle 1 Day 1 to the End of Treatment visit) for an average of 69 days (standard deviation or SD of 54 days), Cohort 10 (5 mg/kg, Q3W) patients stayed on study for an average of 50 days (SD of 39 days), in Cohort 11 (8 mg/kg, Q3W) patients stayed on study for an average of 49 days (SD of 39 days), and in Cohort 11B (8 mg/kg, Q2W) patients stayed on study for an average of 119 days (SD of 9 days). The increasing length of duration on study with increasing doses may correspond to increased serum concentrations of the drug and increased probability and duration of disease response.

### Example 5. Dose escalation study of PRS-343 in patients with HER2+ advanced or metastatic solid tumors

This example provides data for Cohorts 1-13 as well as the obinutuzumab (obi) pre-treatment cohort. Example 4 provides data for Cohorts 1-13, and Example 3 provides data for Cohorts 1-11.

### A. Study Objectives and Overview

The study objectives are as described in Example 3.

Patients are allocated to different dose levels in dedicated Cohorts 1 through 13 and receive PRS-343, as described in Examples 4.

The potential of obinutuzumab pre-treatment to reduce formation of ADA is studied in an up to ten patients receiving PRS-343 at a dose of 8 mg/kg per Schedule 2 (Q2W) (corresponding to Cohort 11). Further doses and schedules with B cell depletion may be tested. If obinutuzumab is shown to reduce ADA formation, and no new safety concerns arise this strategy may be used for B cell depletion and reduction of ADA incidence in further patients receiving PRS-343.

Subject inclusion criteria are as described in Example 3, so as the exclusion criteria, with the addition that: 7. Patients with latent or active hepatitis B infection are excluded from the pre-treatment cohort receiving obinutuzumab; 9. Systemic steroid therapy (>10 mg daily prednisone or equivalent) or any other form of immunosuppressive therapy within 7 days prior to the first dose of study treatment (Note: topical, inhaled, nasal and ophthalmic steroids are not prohibited). This criterion does not apply to patients receiving obinutuzumab as pre-treatment.

### B. Study Procedures

The study procedures are as described in Examples 3 and 4.

For subjects enrolled in obinutuzumab pre-treatment cohorts (receiving PRS-343 Q2W at 8 mg/kg), obinutuzumab is administered according to the GAZYVA® (obinutuzumab) package insert or institutional guidelines.

### C. Endpoints and Assessments

The study procedures are as described in Example 3. For laboratory assessments, hepatitis B virus (HBV) infection is also assessed as active and latent infection with HBV are ruled out before obinutuzumab administration.

### Example 6. Dose escalation study of PRS-343 in patients with HER2+ advanced or metastatic solid tumors

This example provides information on this study for Cohorts 1-13 as well as the obinutuzumab pre-treatment cohort and provides (further) interim data for these cohorts.

### A. Study Objectives and Overview

The study objectives were as described in Examples 3, 4 and 5.

Patients were allocated to different dose levels in dedicated cohorts, as indicated in Table 10, and received PRS-343 administered by intravenous (IV) infusion over 2 hours every 3 weeks (Q3W; dosing on day 1; 21-day cycle), every 2 weeks (Q2W; dosing on days 1 and 15; 28-day cycle) and every week (Q1W; dosing on days 1, 8 and 15; 21-day cycle), respectively.

TABLE 10
Patient cohorts of PRS-343 study

| Cohort | Dose & Regimen |
|---|---|
| 1 | 0.0005 mg/kg Q3W |
| 2 | 0.0015 mg/kg Q3W |
| 3 | 0.005 mg/kg Q3W |
| 4 | 0.015 mg/kg Q3W |
| 5 | 0.05 mg/kg Q3W |
| 6 | 0.15 mg/kg Q3W |
| 7 | 0.5 mg/kg Q3W |
| 8 | 1 mg/kg Q3W |
| 9 | 2.5 mg/kg Q3W |
| 10 | 5 mg/kg Q3W |
| 11 | 8 mg/kg Q3W |
| 11B | 8 mg/kg Q2W |
| 11C | 8 mg/kg Q1W |
| 12B | 12 mg/kg Q2W |
| 13B | 18 mg/kg Q2W |
| Obi + 11B | 8 mg/kg Q2W |

Subject inclusion and exclusion criteria were as described in Example 3. Key inclusion criteria were: diagnosis of HER2+ advanced/metastatic solid tumor malignancy that has progressed on standard therapy or for which no standard therapy is available; HER2+ solid tumors documented by ASCO, CAP or institutional guidelines; patients with breast, gastric and GEJ cancer must have received at least one prior HER2-targeted therapy for advanced/metastatic disease; measurable disease per RECIST v1.1; ECOG 0 or 1; adequate liver, renal, cardiac and bone marrow function. Key exclusion criteria were: ejection fraction below the lower limit of normal with trastuzumab and/or pertuzumab; systemic steroid therapy or any other form of immunosuppressive therapy within seven days prior to registration; known, symptomatic, unstable or progressing CNS primary malignancies; radiation therapy within 21 days prior to registration (limited field radiation to non-visceral structures is allowed, e.g., limb bone metastasis.

### B. Study Procedures

The study procedures were as described in Example 4 (see also Example 5 regarding pre-treatment with obinutuzumab).

### C. Endpoints and Assessments

The study procedures were as described in Examples 3 and 5. In addition, levels of circulating s4-1BB were assessed. s4-1BB has been previously shown to be increased in the sera of patients treated with an anti-4-1BB agonistic monoclonal antibody (Segal et al., 2018).

### D. Data Analysis/Methods

Data analysis and methods were as described in Example 4.

Serum s4-1BB levels were assessed by means of a proprietary enzyme-linked immunosorbent assay (ELISA). An alternative assay for assessing serum s4-1BB levels is described in Segal et al., 2018.

The percentage of PD-L1-positive cells (IC score) was determined by immunohistochemistry (IHC) staining.

### E. Preliminary Results

A total of 74 patients have been treated with PRS-343 administered as a single agent (Tables 10 and 11). The median age at treatment is 63 years and 44 (59%) of the patients were female. 55 (74%) of the treated patients had ECOG PS of 1 and the rest had a PS of 0. This was a previously heavily treated population of patients with 28 or 38% having received 5+ lines of therapy, 11 (15%) having received 4 lines of therapy and 15 or 21% having received 3 lines of therapy. Of the wide range of tumor types studied 27 (36%) had gastroesophageal cancer, 16 (22%) had breast cancer and 10 (14%) had colorectal cancer.

TABLE 11
Baseline characteristics and primary
cancer types of enrolled subjects

| | n (%) | |
|---|---|---|
| Characteristic | | |
| Age, Median (range) | 63 | (24-92) |
| Gender | | |
| F | 44 | (59%) |
| M | 30 | (41%) |
| ECOG PS | | |
| 0 | 19 | (26%) |
| 1 | 55 | (74%) |
| Prior Therapy Lines | | |
| 1 | 9 | (12%) |
| 2 | 10 | (14%) |
| 3 | 15 | (21%) |
| 4 | 11 | (15%) |
| 5+ | 28 | (38%) |
| Average HER2 Targeting Treatments | | |
| Breast | 7 | |
| Gastric | 3 | |
| Primary Cancer Type | | |
| Gastroesophageal | 27 | (36%) |
| Breast | 16 | (22%) |
| Colorectal | 10 | (14%) |
| Gynecological | 9 | (12%) |
| Biliary Tract | 7 | (9%) |
| Bladder | 2 | (3%) |
| Pancreatic | 1 | (1%) |
| Other - Cancer of Unknown Origin | 1 | (1%) |
| Other - Salivary Duct | 1 | (1%) |

Of the treatment related adverse events reported, the most common were infusion related reactions (27 incidents or 19% of all TRAEs), fatigue (11 incidents or 8% of all TRAEs) and nausea in 11 or 8% of all reported TRAEs (Table 12). One TRAE was above grade 3: a grade 4 infusion related reaction in cohort 10 (5 mg/kg PRS-343, Q3W).

TABLE 12
Treatment-related adverse effects (TRAEs)

| Occurred in >1 Patient | N = 145 \| n (%) | % Grade 3 |
|---|---|---|
| Infusion Related Reaction | 27 (19%) | 3 (2%) |
| Fatigue | 11 (8%) | 1 (1%) |
| Nausea | 11 (8%) | 0 |
| Vomiting | 8 (6%) | 0 |
| Chills | 8 (6%) | 0 |
| Anemia | 2 (1%) | 1 (1%) |
| Arthalgia | 2 (1%) | 0 |
| Asthenia | 2 (1%) | 0 |
| Cough | 2 (1%) | 0 |
| Decreased appetite | 2 (1%) | 0 |
| Diarrhea | 6 (4%) | 0 |
| Dizziness | 2 (1%) | 0 |
| Dyspnoea | 3 (2%) | 0 |
| Flushing | 5 (3%) | 2 (1%) |
| Non-cardiac chest pain | 4 (3%) | 0 |
| Paraesthesia | 3 (2%) | 1 (1%) |
| Pruritis | 3 (3%) | 0 |
| Rash | 2 (1%) | 0 |

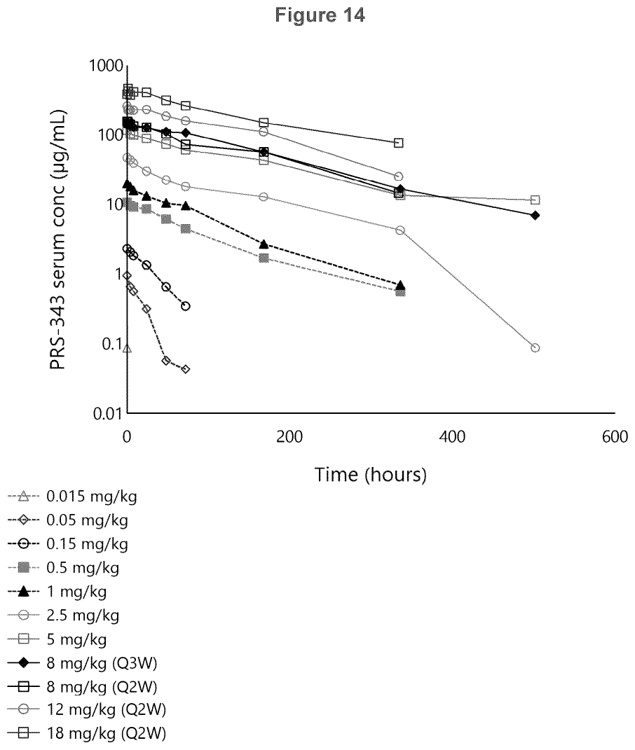

Single dose geometric mean serum concentrations of PRS-343 are shown in FIG. 14. The mean terminal half-life of PRS-343 was approximately five days. 36% of the patients were ADA positive with titers above 1:150 in cohorts covering the active dose range (>2.5 mg/kg) (data not shown).

Based on clinical data in the study population, it was estimated that 20 pg/mL is the serum concentration of the drug which results in an efficacious dose in the tumor microenvironment. This serum concentration was reached in Cohort 9. 33 evaluable patients are present in Cohorts 9-13B, of which 1 patient recorded a complete response, 3 patients recorded a partial response and 13 patients showed stable disease (Table 13).

TABLE 13
Summary of Response at Active Dose Range of PRS-343

| | 13B | 12B | 11C | Obi | 11B | 11 | 10 | 9 | |
|---|---|---|---|---|---|---|---|---|---|
| Cohort | 18 | 12 | 8 | 8 | 8 | 8 | 5 | 2.5 | |
| Best | mg/kg, | mg/kg, | mg/kg, | mg/kg, | mg/kg, | mg/kg, | mg/kg, | mg/kg, | |
| Response | Q2W | Q2W | Q1W | Q2W | Q2W | Q3W | Q3W | Q3W | Total |
| Evaluable | 3 | 2 | 4 | 2 | 7 | 4 | 6 | 5 | 33 |
| Patients | | | | | | | | | |
| CR | 1 | — | — | — | — | — | — | — | 1 |
| PR | — | — | — | — | 3 | . | — | — | 3 |
| SD | — | — | 1 | 1 | 3 | 3 | 3 | 2 | 13 |
| ORR | 33% | 0% | 0% | 0% | 43% | 0% | 0% | 0% | 12% |
| DCR | 33% | 0% | 25% | 50% | 86% | 75% | 50% | 40% | 52% |

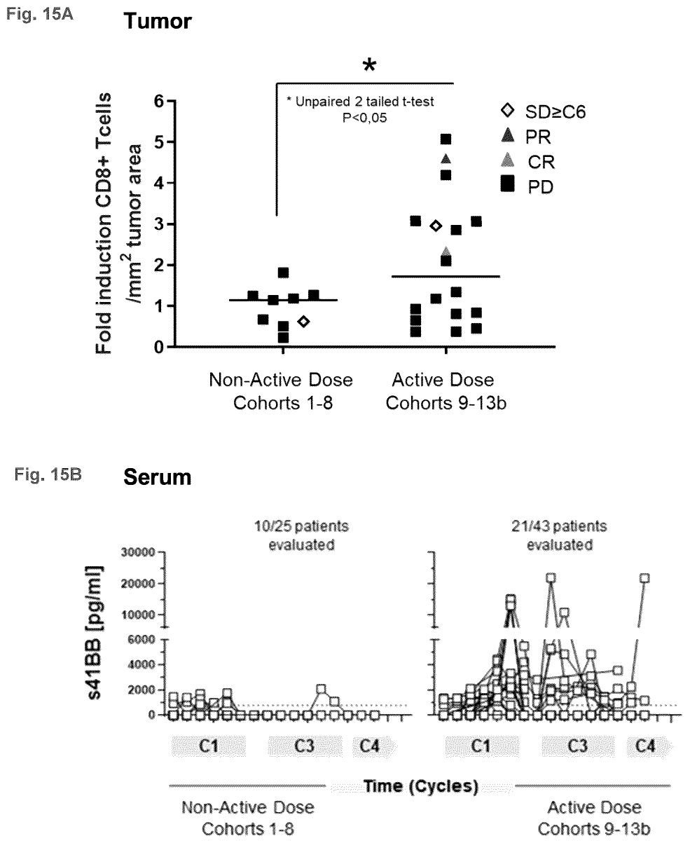

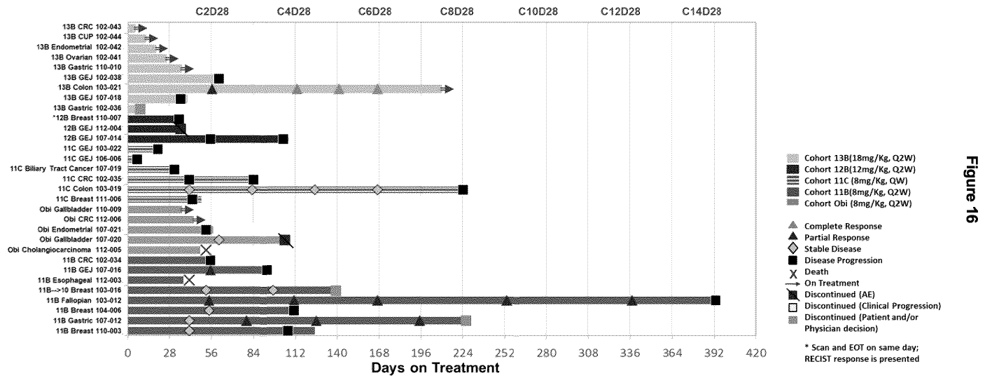

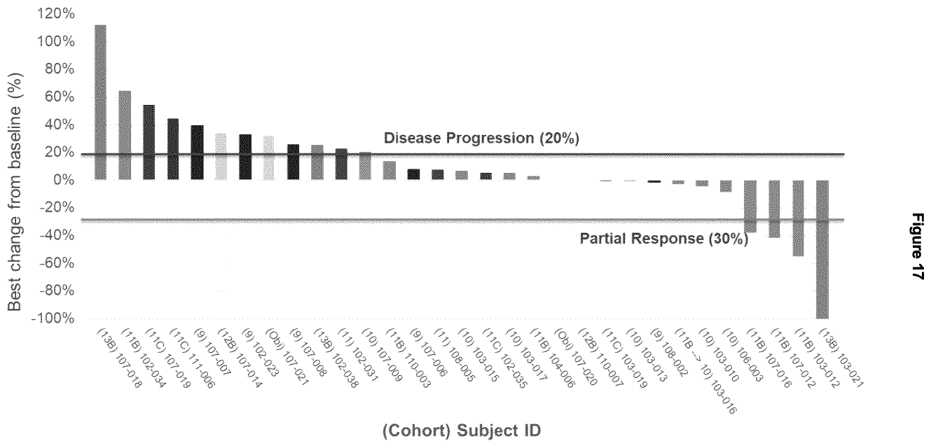

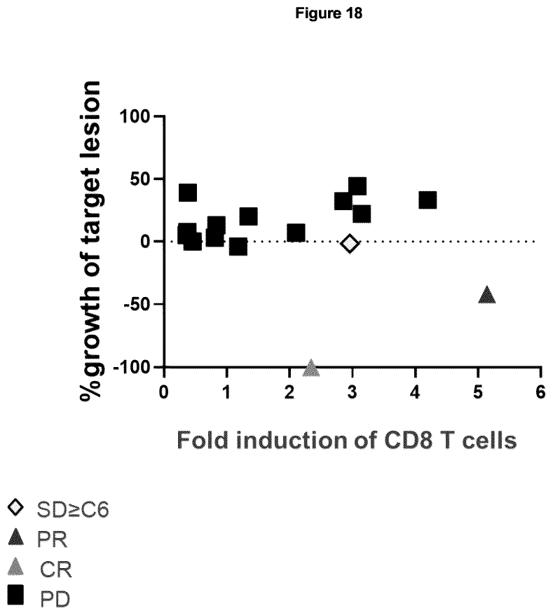

Pre-dose biopsies and post-dose biopsies (cycle 2; days 2-8) were performed. As shown in FIG. 15A, patients treated with active doses of PRS-343 (Cohorts 9-13B) showed increased CD8+ T cells in the tumor tissue. Furthermore, these patient exhibited increased levels of circulating s4-1BB in the serum (FIG. 15B), demonstrating 4-1 BB arm activity of PRS-343. The course of treatment for patients in Cohorts 11B, 11C, 12B, 13B and Obi+11B over time, including the clinical status (where applicable), such as complete response, partial response, stable disease and disease progression, is shown in FIG. 16. FIG. 17 shows the best response in target lesions for Cohorts 9, 10, 11, 11B, 11C, 12B, 13B and Obi+11B. As shown in FIG. 18, patients with prolonged clinical benefit (SD2C6, PR and CR) exhibited an increase of CD8+ T cells in full tumor tissue.

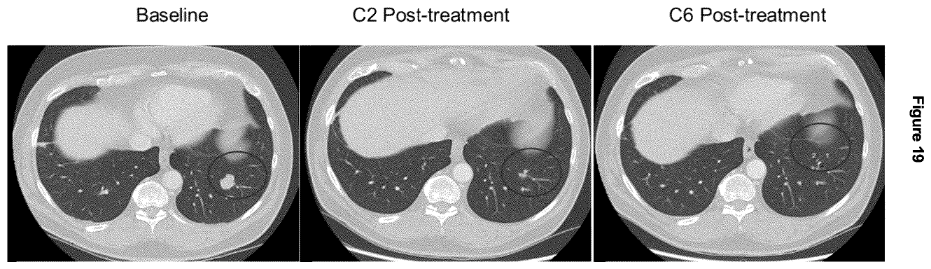

As shown in Tables 13 and 14 as well as FIGS. 16 to 19, one patient of Cohort 13B (18 mg/kg, Q2W) exhibited a complete response upon treatment with PRS-343 (see, in particular, CT scans depicted in FIG. 19). The patient is a 59-year old male with stage 4 rectal adenocarcinoma cancer which had metastasized to the heart and lung (prior therapy lines: 5+; FoundationOne HER2 amplification, in-house testing IHC3+; MSS, TMB low (2 mt/Mb)).

TABLE 14
Rectal cancer patient with confirmed CR

| | | Lesion size (mm) | | | |
|---|---|---|---|---|---|
| | Lesion | | C2 Post- | C4 Post- | C6 Post- |
| Lesions | Site | Baseline | treatment | treatment | treatment |
| Target 1 | Lung | 22 | 13 | 0 | 0 |
| % Change | — | — | −41% | −100% | −100% |
| from | | | | | |
| baseline | | | | | |
| Non-target 1 | — | Present | Present | Absent | Absent |

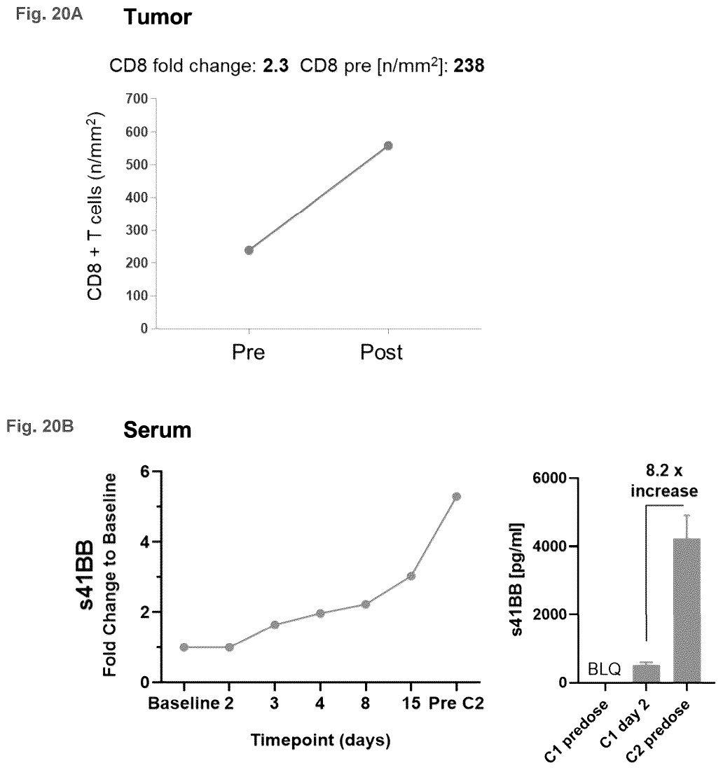

As shown in FIG. 20, post-treatment the patient exhibited increased CD8+ T cell numbers in the tumor (FIG. 20A) and increased circulating s4-1BB levels in the serum, demonstrating 4-1BB arm activity of PRS-343 (FIG. 20B).

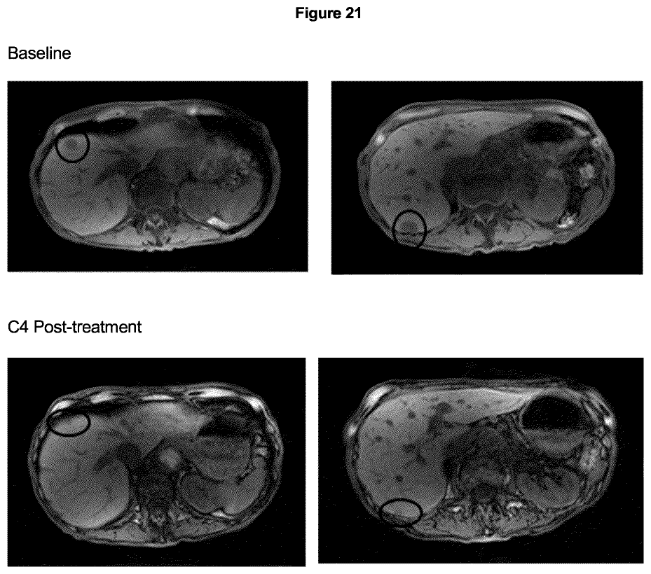

Table 15 shows the treatment outcome for a gastric cancer patient (107-012) of cohort 11B (8 mg/kg, Q2W) with confirmed partial response (see also CT scans in FIG. 21). The patient is an 80-year old woman with stage 4 gastric adenocarcinoma which had metastasized to the liver, lymph node and adrenal glands (prior therapy lines: 2; HER2 IHC3+; PD-L1-positive (CPS=3); NGS: ERBB2 amplification, TP53 mutation, alteration of CDK12 and SF3B1).

TABLE 15
Gastric cancer patient with confirmed PR

| | | Lesion Size (mm) | | | | |
|---|---|---|---|---|---|---|
| | Lesion | | C2 Post- | C3 Post- | C4 Post- | C6 Post- |
| Lesions | Site | Baseline | treatment | treatment | treatment | treatment |
| Target 1 | Liver | 14 | 12 | 10 | 9 | 8 |
| Target 2 | Liver | 20 | 16 | 10 | 8 | 9 |
| Target 3 | Pancreas | 19 | 16 | 14 | 14 | 14 |
| % Change | — | — | −17% | −36% | −42% | −42% |
| from | | | | | | |
| baseline | | | | | | |
| Non-target | Lung | Present | Present | Present | Present | Present |
| 1 | | | | | | |
| Non-target | Stomach | Present | Present | Present | Present | Absent |
| 2 | | | | | | |
| Non-target | Stomach | Present | Present | Present | Present | Absent |
| 3 | | | | | | |

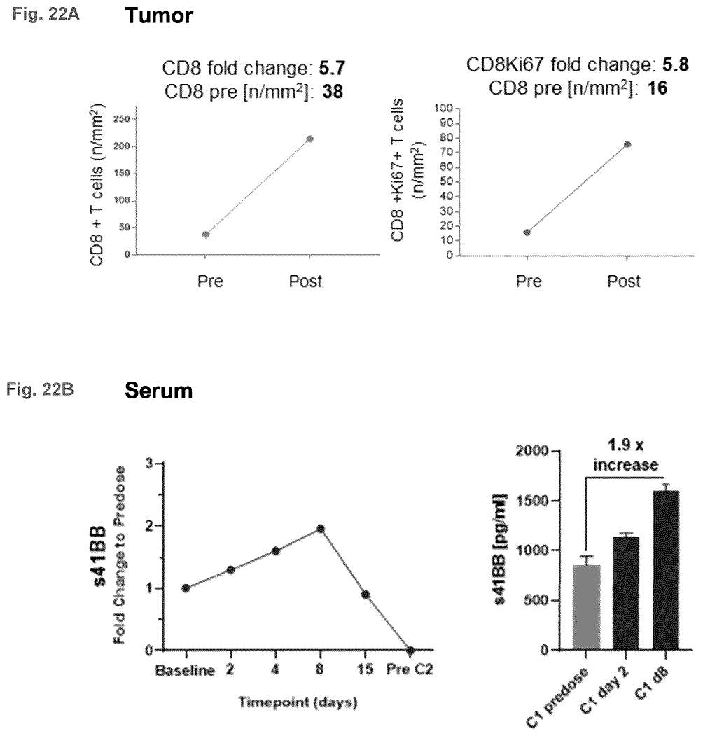

As shown in FIG. 22, post-treatment the patient exhibited increased 008+ T cell numbers and CD8+Ki67+ T cell numbers in the tumor (FIG. 22A) as well as increased circulating s4-1 BB levels in the serum, demonstrating 4-1 BB arm activity of PRS-343 (FIG. 22B).

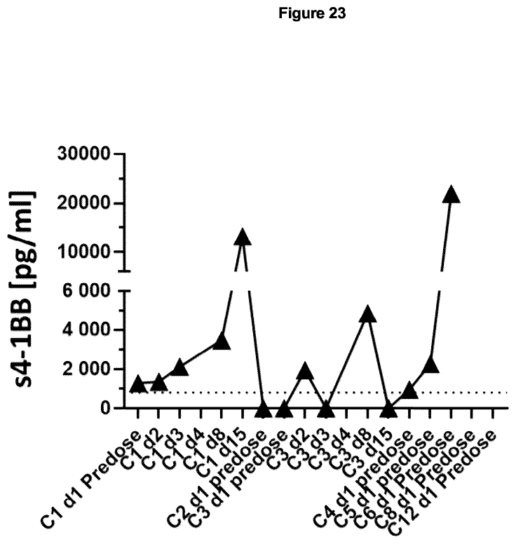

FIG. 23 shows a repeated increase of circulating s4-1 BB in the serum of the PR patient 103-012 of cohort 11 B (8 mg/kg, Q2VV) over the course of multiple treatment cycles. The patient has fallopian tube cancer.

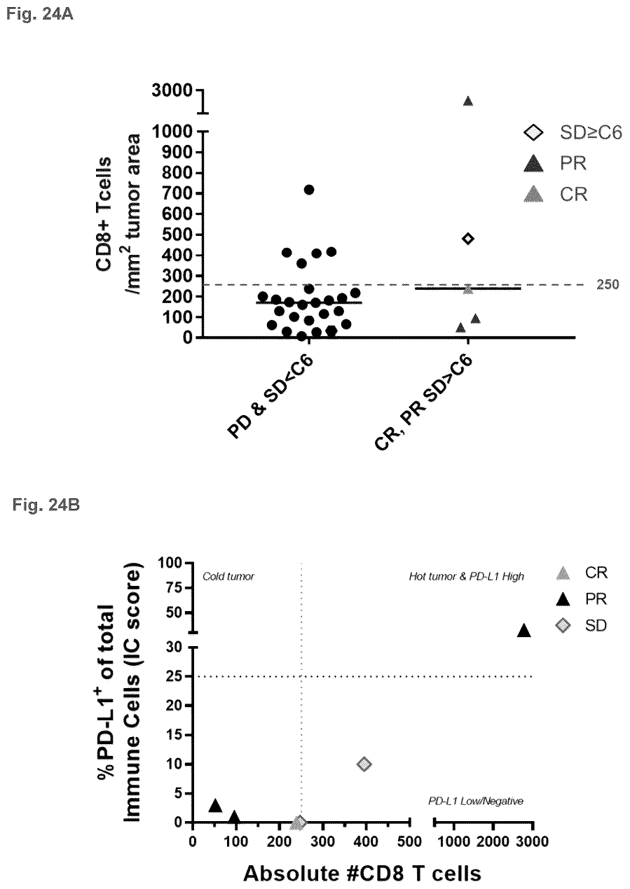

FIG. 24 shows that PRS-343 drives prolonged clinical benefit (including partial response and complete response) in patients with low 008+ T cell counts prior to therapy (<250/mm² tumor area; FIGS. 24A and B) as well as in PD-L1 low/negative patients (<25% PD-L1+ cells of total immune cells (IC score); FIG. 24B).

FIG. 25 shows dose dependency of serum levels of s4-1 BB (measured over the course of cycle 1) upon treatment with PRS-343 across all tested dose cohorts, indicating that the s4-1 BB levels can be used to assess the dose-dependent activity of a 4-1 BB agonistic agent, such as PRS-343.

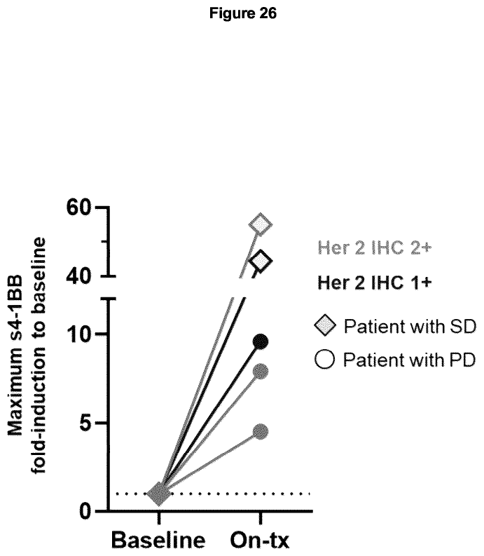

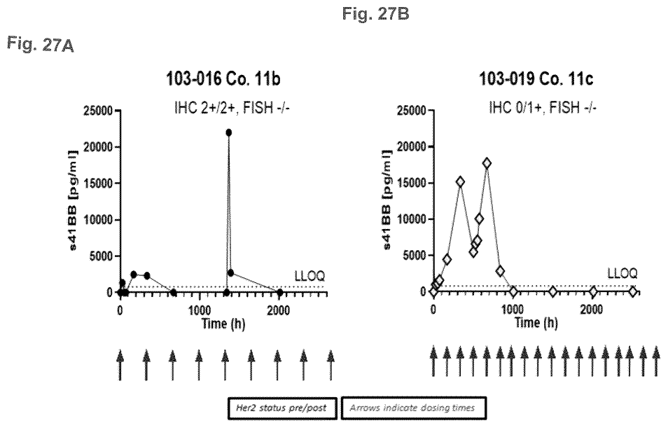

Based on testing of HER2 on fresh biopsies, five patients were identified that, in contrast to archival tissue assessment, were HER2 low. As shown in FIG. 26, all five patients exhibited increased s4-1BB serum levels—however, the two patients clinically benefiting from the treatment with PRS-343, breast cancer patient 103-016 (stable disease at cycles 2 and 4) and colorectal cancer patient 103-019 (stable disease at cycles 2, 4 and 6), showed a significantly higher maximum fold-induction (>40-fold) than the three patients with progressive disease. This indicates a correlation between the extent of s4-1BB induction and clinical response. FIGS. 27A and B show the s4-1BB serum profiles of patients 103-016 and 103-109, respectively.

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

Equivalents: Those skilled in the art will recognize or be able to ascertain using no more than routine experimentation, many equivalents to the specific embodiments of the invention described herein. Such equivalents are intended to be encompassed by the following claims. All publications, patents and patent applications mentioned in this specification are herein incorporated by reference into the specification to the same extent as if each individual publication, patent or patent application was specifically and individually indicated to be incorporated herein by reference.

### VII. NON-PATENT REFERENCES

1. ALTSCHUL, S. F., GISH, W., MILLER, W., MYERS, E. W. & LIPMAN, D. J. 1990. Basic local alignment search tool. J Mol Biol, 215, 403-10.
2. ALTSCHUL, S. F., MADDEN, T. L., SCHAFFER, A. A., ZHANG, J., ZHANG, Z., MILLER, W. & LIPMAN, D. J. 1997. Gapped BLAST and PSI-BLAST: a new generation of protein database search programs. Nucleic Acids Res, 25, 3389-402.
3. BARTLEY, A. N., WASHINGTON, M. K., VENTURA, C. B., ISMAILA, N., COLASACCO, C., BENSON III, A. B., CARRATO, A., GULLEY, M. L., JAIN, D., KAKAR, S., MACKAY, H. J., STREUTKER, C., TANG, L., TROXELL, M., AJANI, J. A. 2016. HER2 testing and clinical decision making in gastroesophageal adenocarcinoma. Arch Pathol Lab Med, 140, 1345-63.
4. BLANDO, J., SHARMA, A., HIGA, M. G., ZHAO, H., VENCE, L., YADAV, S. S., KIM, J., SEPULVEDA, A. M., SHARP, M., MAITRA, A., WARGO, J., TETZLAFF, M., BROADDUS, R., KATZ, M. H. G., VARADHACHARY, G. R., OVERMAN, M., WANG, H., YEE, C., BERNATCHEZ, C., IACOBUZIO-DONAHUE, C., BASU, S., ALLISON, J. P. & SHARMA, P. 2019. Comparison of immune infiltrates in melanoma and pancreatic cancer highlights VISTA as a potential target in pancreatic cancer. Proc Nat/Acad Sci USA, 116, 1692-1697.
5. CHEN, P. L., ROH, W., REUBEN, A., COOPER, Z. A., SPENCER, C. N., PRIETO, P. A., MILLER, J. P., BASSETT, R. L., GOPALAKRISHNAN, V., WANI, K., DE MACEDO, M. P., AUSTIN-BRENEMAN, J. L., JIANG, H., CHANG, Q., REDDY, S. M., CHEN, W. S., TETZLAFF, M. T., BROADDUS, R. J., DAVIES, M. A., GERSHENWALD, J. E., HAYDU, L., LAZAR, A. J., PATEL, S. P., HWU, P., HWU, W. J., DIAB, A., GLITZA, I. C., WOODMAN, S. E., VENCE, L. M., WISTUBA, II, AMARIA, R. N., KWONG, L. N., PRIETO, V., DAVIS, R. E., MA, W., OVERWIJK, W. W., SHARPE, A. H., HU, J., FUTREAL, P. A., BLANDO, J., SHARMA, P., ALLISON, J. P., CHIN, L. & WARGO, J. A. 2016. Analysis of Immune Signatures in Longitudinal Tumor Samples Yields Insight into Biomarkers of Response and Mechanisms of Resistance to Immune Checkpoint Blockade. Cancer Discov, 6, 827-37.
6. DALL′ACQUA, W. F., KIENER, P. A. & WU, H. 2006. Properties of human IgG1s engineered for enhanced binding to the neonatal Fc receptor (FcRn). J Biol Chem, 281, 23514-24.
7. DAWICKI, W. & WATTS, T. H. 2004. Expression and function of 4-1BB during CD4 versus CD8 T cell responses in vivo. Eur J Immunol, 34, 743-751.
8. EISENHAUER, E. A., THERASSE, P., BOGAERTS, J., SCHWARTZ, L. H., SARGENT, D., FORD, R., DANCEY, J., ARBUCK, S., GWYTHER, S., MOONEY, M., RUBINSTEIN, L., SHANKAR, L., DODD, L., KAPLAN, R., LACOMBE, D. & VERWEIJ, J. 2009. New response evaluation criteria in solid tumours: revised RECIST guideline (version 1.1). EurJ Cancer, 45, 228-47.
9. FLOWER, D. R. 1996. The lipocalin protein family: structure and function. Biochem J, 318 (Pt 1), 1-14.
10. FLOWER, D. R., NORTH, A. C. & SANSOM, C. E. 2000. The lipocalin protein family: structural and sequence overview. Biochim Biophys Acta, 1482, 9-24.
11. GLAESNER, W., VICK, A. M., MILLICAN, R., ELLIS, B., TSCHANG, S. H., TIAN, Y., BOKVIST, K., BRENNER, M., KOESTER, A., PORKSEN, N., ETGEN, G. & BUMOL, T. 2010. Engineering and characterization of the long-acting glucagon-like peptide-1 analogue LY2189265, an Fc fusion protein. Diabetes Metab Res Rev, 26, 287-96.
12. HOLLIGER, P., PROSPERO, T. & WINTER, G. 1993. “Diabodies”: small bivalent and bispecific antibody fragments. Proc Nat/Acad Sci USA, 90, 6444-8.
13. LEE, H. W., PARK, S. J., CHOI, B. K., KIM, H. H., NAM, K. O. & KWON, B. S. 2002. 4-1BB promotes the survival of CD8+T lymphocytes by increasing expression of Bcl-xL and Bfl-1. J Immunol, 169, 4882-8.
14. NEEDLEMAN, S. B. & WUNSCH, C. D. 1970. A general method applicable to the search for similarities in the amino acid sequence of two proteins. J Mol Biol, 48, 443-53.
15. PEARSON, W. R. & LIPMAN, D. J. 1988. Improved tools for biological sequence comparison. Proc Nat/Acad Sci USA, 85, 2444-8.
16. SEGAL, N. H., HE, A. R., DOI, T., LEVY, R., BHATIA, S., PISHVAIAN, M. J., CESARI, R., CHEN, Y., DAVIS, C. B., HUANG, B., THALL, A. D. & GOPAL, A. K. 2018. Phase I Study of Single-Agent Utomilumab (PF-05082566), a 4-1BB/CD137 Agonist, in Patients with Advanced Cancer. Clin Cancer Res, 24, 1816-1823.
17. SILVA, J. P., VETTERLEIN, O., JOSE, J., PETERS, S. & KIRBY, H. 2015. The S228P mutation prevents in vivo and in vitro IgG4 Fab-arm exchange as demonstrated using a combination of novel quantitative immunoassays and physiological matrix preparation. J Biol Chem, 290, 5462-9.
18. SKERRA, A. 2000. Lipocalins as a scaffold. Biochim Biophys Acta, 1482, 337-50.
19. SMITH, T. F. & WATERMAN, M. S. 1981. Identification of common molecular subsequences. J Mol Biol, 147, 195-7.
20. SNELL, L. M., LIN, G. H., MCPHERSON, A. J., MORAES, T. J. & WATTS, T. H. 2011. T-cell intrinsic effects of GITR and 4-1 BB during viral infection and cancer immunotherapy. Immunol Rev, 244, 197-217.
21. TUMEH, P. C., HARVIEW, C. L., YEARLEY, J. H., SHINTAKU, I. P., TAYLOR, E. J., ROBERT, L., CHMIELOWSKI, B., SPASIC, M., HENRY, G., CIOBANU, V., WEST, A. N., CARMONA, M., KIVORK, C., SEJA, E., CHERRY, G., GUTIERREZ, A. J., GROGAN, T. R., MATEUS, C., TOMASIC, G., GLASPY, J. A., EMERSON, R. O., ROBINS, H., PIERCE, R. H., ELASHOFF, D. A., ROBERT, C. & RIBAS, A. 2014. PD-1 blockade induces responses by inhibiting adaptive immune resistance. Nature, 515, 568-71.
22. WARD, E. S., GUSSOW, D., GRIFFITHS, A. D., JONES, P. T. & WINTER, G. 1989. Binding activities of a repertoire of single immunoglobulin variable domains secreted from Escherichia coli. Nature, 341, 544-6.
23. WOLFF, A. C., HALE HAMMOND, E., ALLISON, K. H., HARVEY, B. E., MANGU, P. B., BARTLETT, J., BILOUS, M., ELLIS, I. O., FITZGIBBONS, P., HANNA, W., JENKINS, R. B., PRESS, M. F., SPEARS, P. A., VANCE, G. H., VIALE, G., MCSHANE, L. M., DOWSETT, M. 2018. Human epidermal growth factor receptor 2 testing in breast cancer. Arch Pathol Lab Med, 142, 1364-82.
24. YAO, S., ZHU, Y. & CHEN, L. 2013. Advances in targeting cell surface signalling molecules for immune modulation. Nat Rev Drug Discov, 12, 130-46.
25. ZALEVSKY, J., CHAMBERLAIN, A. K., HORTON, H. M., KARKI, S., LEUNG, I. W., SPROULE, T. J., LAZAR, G. A., ROOPENIAN, D. C. & DESJARLAIS, J. R. 2010. Enhanced antibody half-life improves in vivo activity. Nat Biotechnol, 28, 157-9.

1. A method of predicting a positive clinical outcome for a cancer patient upon treatment with a 4-1BB agonistic agent, said method comprising (a) measuring the level of soluble 4-1BB (s4-1BB) in a biological sample obtained from the cancer patient prior to administering the 4-1BB agonistic agent to the cancer patient; and (b) measuring the level of s4-1BB in a biological sample obtained from the cancer patient after administering the 4-1BB agonistic agent to the cancer patient, wherein a positive clinical outcome is predicted if the level of s4-1BB in the biological sample obtained from the cancer patient after administering the 4-1BB agonistic agent to the cancer patient is increased as compared to the level of s4-1BB in the biological sample obtained from the cancer patient prior to administering the 4-1BB agonistic agent to the cancer patient.

2. The method of claim 1, wherein the positive clinical outcome comprises stable disease (SD), partial response (PR), complete response (CR), increased overall survival (OS) and/or increased progression free survival (PFS).

3. A method of assessing activity of a 4-1BB agonistic agent in a cancer patient treated with the 4-1BB agonistic agent, said method comprising (a) measuring the level of s4-1BB in a biological sample obtained from the cancer patient prior to administering the 4-1BB agonistic agent to the cancer patient; and (b) measuring the level of s4-1BB in a biological sample obtained from the cancer patient after administering the 4-1BB agonistic agent to the cancer patient, wherein a level of s4-1BB in the biological sample obtained from the cancer patient after administering the 4-1BB agonistic agent to the cancer patient which is increased as compared to the level of s4-1BB in the biological sample obtained from the cancer patient prior to administering the 4-1BB agonistic agent to the cancer patient indicates activity of the 4-1BB agonistic agent in the cancer patient.

4. The method of claim 3, wherein the activity is dose-dependent activity.

5. A method of treating a cancer patient comprising administering an effective amount of a 4-1BB agonistic agent to the cancer patient, said method comprising the steps: (a) measuring the level of s4-1BB in a biological sample obtained from the cancer patient prior to administering the 4-1BB agonistic agent to the cancer patient; (b) administering the 4-1BB agonistic agent to the cancer patient; (c) measuring the level of s4-1BB in a biological sample obtained from the cancer patient after administering the 4-1BB agonistic agent to the cancer patient; and (d) continuing to administer the 4-1BB agonistic agent to the cancer patient, if the level of s4-1BB in the biological sample obtained from the cancer patient after administering the 4-1BB agonistic agent to the cancer patient is increased as compared to the level of s4-1BB in the biological sample obtained from the cancer patient prior to administering the 4-1BB agonistic agent to the cancer patient.

6. The method of claim 5, wherein administration of the 4-1BB agonistic agent to the cancer patient is discontinued if the level of s4-1BB in the biological sample obtained from the cancer patient after administering the 4-1BB agonistic agent to the cancer patient is not increased as compared to the level of s4-1BB in the biological sample obtained from the cancer patient prior to administering the 4-1BB agonistic agent to the cancer patient.

7. A method of selecting a dose of a 4-1BB agonistic agent for treating a disease, e.g., cancer, said method comprising (a) measuring the level of s4-1BB in biological samples obtained from a plurality of subjects having the disease upon administration of different doses of the 4-1BB agonistic agent, and (b) generating a dose response curve based on the results obtained in step (a), wherein, if the level of s4-1BB decreases at a dose X, a dose which is lower than dose X is selected as the dose for treating the disease.

8. The method of claim 7, wherein the decrease of the level of s4-1BB at dose X indicates an overactivation of the 4-1BB pathway or a potential for overactivation of the 4-1BB pathway.

9. The method of claim 7 or 8, wherein the dose is a maintenance dose which is administered after administration of an initial higher dose.

10. The method of any one of claims 1-9, wherein the biological sample is blood serum or blood plasma.

11. The method of any one of claims 1-10, wherein the level of s4-1BB in the biological sample obtained from the cancer patient after administering the 4-1BB agonistic agent is increased by at least about 1.1-fold, at least about 1.2-fold, at least about 1.3-fold, at least about 1.4-fold, at least about 1.5-fold, at least about 2-fold, at least about 2.5-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold or even more fold, as compared to the level of s4-1BB in the biological sample obtained from the cancer patient prior to administering the 4-1BB agonistic agent to the cancer patient.

12. The method of any one of claims 1-11, wherein the level of s4-1BB in the biological sample obtained from the cancer patient after administering the 4-1BB agonistic agent is increased by about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml of the biological sample, as compared to the level of s4-1BB in the biological sample obtained from the cancer patient prior to administering the 4-1BB agonistic agent to the cancer patient.

13. The method of any one of claims 1-12, wherein the level of s4-1BB in the biological sample obtained from the cancer patient after administering the 4-1BB agonistic agent is increased to a concentration of about 500 or more, about 1000 or more, about 2000 or more, about 3000 or more, about 4000 or more, about 5000 or more, about 6000 or more, about 7000 or more, about 8000 or more, about 9000 or more, about 10000 or more, about 15000 or more, or about 20000 or more pg/ml of the biological sample.

14. The method of any one of claims 1-13, wherein measuring the level of s4-1BB in a biological sample obtained from the cancer patient after administering the 4-1BB agonistic agent to the cancer patient comprises measuring the levels of s4-1BB during and/or after multiple (e.g., two, three, four, or more) cycles of treatment with the 4-1BB agonistic agent.

15. The method of any one of claims 1-14, wherein the level of s4-1BB in the biological sample obtained from the cancer patient after administering the 4-1BB agonistic agent to the cancer patient is increased as compared to the level of s4-1BB in the biological sample obtained from the cancer patient prior to administering the 4-1BB agonistic agent to the cancer patient during or after at least one, at least two, at least three, or at least four cycle(s) of treatment with the 4-1BB agonistic agent.

16. The method of claim 14 or 15, wherein the level of s4-1BB in the biological sample obtained from the cancer patient after administering the 4-1BB agonistic agent to the cancer patient is repeatedly increased as compared to the level of s4-1BB in the biological sample obtained from the cancer patient prior to administering the 4-1BB agonistic agent to the cancer patient during or after multiple (e.g., two, three, four, or more) cycles of treatment with the 4-1BB agonistic agent.

17. The method of any one of claims 14-16, wherein the level of s4-1BB in the biological sample obtained from the cancer patient after administering the 4-1BB agonistic agent to the cancer patient is increased as compared to the level of s4-1BB in the biological sample obtained from the cancer patient prior to administering the 4-1BB agonistic agent to the cancer patient during or after each of the multiple (e.g., two, three, four, or more) cycles of treatment with the 4-1BB agonistic agent.

18. The method of any one of claims 14-17, wherein the cycle of treatment with the 4-1BB agonistic agent comprises: (i) about 21 days, wherein the 4-1BB agonistic agent is administered at an interval of about once every three weeks (Q3W); (ii) about 28 days, wherein the 4-1BB agonistic agent is administered at an interval of about once every two weeks (Q2WA); or (iii) about 21 days, wherein the 4-1BB agonistic agent is administered at an interval of about once every week (Q1WA).

19. The method of any one of claims 1-18, wherein the level of s4-1BB in the biological sample obtained from the cancer patient after administering the 4-1BB agonistic agent to the cancer patient is the maximum level of s4-1BB measured during and/or after one or more (e.g., two, three, four, or more) cycles of treatment with the 4-1BB agonistic agent.

20. The method of claim 19, wherein the maximum level of s4-1BB measured during and/or after one or more (e.g., two, three, four, or more) cycles of treatment with the 4-1BB agonistic agent is increased by at least about 15, at least about 20, at least about 25, at least about 30, at least about 35, at least about 40 or even more folds, as compared to the level of s4-1BB in the biological sample obtained from the cancer patient prior to administering the 4-1BB agonistic agent to the cancer patient.

21. The method of any one of claims 1-18, wherein the level of s4-1BB in the biological sample obtained from the cancer patient after administering the 4-1BB agonistic agent to the cancer patient is the average level of s4-1BB measured during and/or after one or more (e.g., two, three, four, or more) cycles of treatment with the 4-1BB agonistic agent.

22. Use of s4-1BB as a predictive biomarker for the clinical outcome of a cancer patient upon treatment with a 4-1BB agonistic agent.

23. Use of s4-1BB as a biomarker for activity, preferably dose-dependent activity, of a 4-1BB agonistic agent in a cancer patient treated with the 4-1BB agonistic agent.

24. Use of s4-1BB as a biomarker for selecting a dose of a 4-1BB agonistic agent for treating a disease, e.g., cancer.

25. Use of a kit comprising means for detecting s4-1BB in a biological sample for predicting a positive clinical outcome for a cancer patient upon treatment with a 4-1BB agonistic agent.

26. Use of a kit comprising means for detecting s4-1BB in a biological sample for assessing activity, preferably dose-dependent activity, of a 4-1BB agonistic agent in a cancer patient treated with the 4-1BB agonistic agent.

27. Use of a kit comprising means for detecting s4-1BB in a biological sample for selecting a dose of a 4-1BB agonistic agent for treating a disease, e.g., cancer.

28. The use of any one of claims 25-27, wherein the biological sample is blood serum or blood plasma.

29. The use of any one of claims 25-28, wherein the means for detecting s4-1BB in a biological sample comprise an antibody specific for 4-1BB and/or s4-1BB.

30. The use of any one of claims 25-29, wherein the kit is an immunoassay kit.

31. The use of any one of claims 25-30, wherein the kit further comprises one or more of the following: a container containing a diluent, a container containing a buffer, a container containing an enzyme-conjugate, a container containing a substrate solution, a container containing a secondary antibody, a container containing beads, a multi-well plate, a data carrier.

32. The method or use of any one of claims 1-31, wherein the 4-1BB agonistic agent comprises a lipocalin mutein specific for 4-1BB.

33. The method or use of claim 32, wherein the lipocalin mutein comprises the amino acid sequence shown in SEQ ID NO: 22 or an amino acid sequence having at least 95% sequence identity to the amino acid sequence shown in SEQ ID NO: 22.

34. The method or use of any one of claims 1-33, wherein the 4-1BB agonistic agent is part of a fusion molecule comprising the 4-1BB agonistic agent and a tumor-targeting moiety.

35. The method or use of claim 34, wherein the tumor-targeting moiety is specific for a tumor antigen expressed on the surface of a tumor cell.

36. The method or use of claim 35, wherein the tumor antigen is HER2.

37. The method or use of any one of claims 1-36, wherein the cancer is characterized by a low expression of HER2.

38. The method or use of claim 37, wherein the cancer is characterized by a HER2 status of IHC1+ or IHC2+/(F)ISH−.

39. The method or use of any one of claims 34-38, wherein the tumor-targeting moiety comprises an antibody or an antigen-binding fragment thereof.

40. The method or use of any one of claims 34-39, wherein the fusion molecule is a fusion protein comprising an antibody specific for a tumor antigen expressed on the surface of a tumor cell fused at the C-terminus of both heavy chains to the N-terminus of a lipocalin mutein specific for 4-1BB.

41. The method or use of claim 39 or 40, wherein the antibody is specific for HER2 and comprises: (i) three heavy chain complementarity-determining regions (CDRs) shown in SEQ ID NO: 40, SEQ ID NO: 41, and SEQ ID NO: 42, and three light chain CDRs shown in SEQ ID NO: 43, SEQ ID NO: 44, and SEQ ID NO: 45; and (ii) a heavy chain with at least 95% sequence identity to the amino acid sequence shown in SEQ ID NO: 49, and a light chain with at least 95% sequence identity to an amino acid sequence shown in SEQ ID NO: 50.

42. The method or use of any one of claims 34-41, wherein the fusion molecule is a fusion protein comprising an antibody specific for HER2 fused at the C-terminus of both heavy chains to the N-terminus of a lipocalin mutein specific for 4-1BB.

43. The method or use of claim 42, wherein the fusion protein has at least 95% sequence identity to the amino acid sequences shown in SEQ ID NOs: 50 and 51.

44. The method or use of claim 42 or 43, wherein the fusion protein comprises the amino acid sequences shown in SEQ ID NOs: 50 and 51.

45. The method or use of any one of claims 42-44, wherein the fusion protein comprises two chains having the amino acid sequence shown in SEQ ID NO: 50 and two chains having the amino acid sequence shown in SEQ ID NO: 51.
